SEQUENCE LISTING (15 sequences)

SEQ ID NO: 1 - RNA, 21 nt, Artificial Sequence
  Guide strand of siRNA n1
ugauaguagucauaaucgcua

SEQ ID NO: 2 - RNA, 21 nt, Artificial Sequence
  Passenger strand of siRNA n1
gcgauuaugacuacuauuuua

SEQ ID NO: 3 - RNA, 21 nt, Artificial Sequence
  Guide strand of siRNA n2
ugacuuaaaguucuuuaugcg

SEQ ID NO: 4 - RNA, 21 nt, Artificial Sequence
  Passenger strand of siRNA n2
cauaaagaacuuuaaguccuc

SEQ ID NO: 5 - RNA, 21 nt, Artificial Sequence
  Guide strand of siRNA n3
uuagcuaaagacacgaaccgg

SEQ ID NO: 6 - RNA, 21 nt, Artificial Sequence
  Passenger strand of siRNA n3
gguucgugucuuuagcuacuc

SEQ ID NO: 7 - DNA, 31703 bp, Artificial Sequence
  Nucleic acid sequence of SARS-CoV-2 virus genome
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacattaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 8 - RNA, 22 nt, Artificial Sequence
  guide strand of siRNA N4
ugacuuaaaguucuuuaugcuc

SEQ ID NO: 9 - RNA, 22 nt, Artificial Sequence
  passenger strand of siRNA N4
gcauaaagaacuuuaaguuucu

SEQ ID NO: 10 - RNA, 21 nt, Artificial Sequence
  guide strand
uauagcuaaagacacgaaccc

SEQ ID NO: 11 - RNA, 21 nt, Artificial Sequence
  guide strand
auagcuaaagacacgaaccgg

SEQ ID NO: 12 - RNA, 21 nt, Artificial Sequence
  guide strand
uugagugcaucauuauccaag

SEQ ID NO: 13 - RNA, 21 nt, Artificial Sequence
  guide strand
cuugacugccgccucugcucg

SEQ ID NO: 14 - RNA, 21 nt, Artificial Sequence
  guide strand
guugagugcaucauuauccac

SEQ ID NO: 15 - RNA, 21 nt, Artificial Sequence
  guide strand
uccugauuauguacaacaccg

(19) United States
(12) Patent Application Publication
(10) Pub. No.: US 2023/0144739 A1
(43) Pub. Date: May 11, 2023

(54) TREATMENT OF COVID-19 WITH REVERSE MICELLE SYSTEM COMPRISING UNMODIFIED OLIGONUCLEOTIDES

(71) Applicants: Medesis Pharma, Baillargues (FR); Centre National de la Recherche Scientifique, Paris (FR); Universite de Montpellier, Montpellier (FR)

(72) Inventors: Jean-Claude MAUREL, Baillargues (FR); Abdelkader MOURI, Montpellier (FR); Hervé SEITZ, Grabels (FR); Sophie MOCKLY, Saint Amand Montrond (FR)

(21) Appl. No.: 17/906,500

(22) PCT Filed: Mar. 15, 2021
(86) PCT No.: PCT/EP2021/056541

§ 371 Date: Sep. 16, 2022

(30) Foreign Application Priority Data
Mar. 16, 2020 (EP) 20305272.5

(51) Int. Cl. A61K 9/107; A61K 47/28; A61K 47/24; A61K 47/10; A61K 47/26; C12N 15/113; A61P 31/14; A61K 9/00

(52) U.S. Cl. CPC A61K 9/1075; A61K 47/28; A61K 47/24; A61K 47/10; A61K 47/26; C12N 15/1131; A61P 31/14; A61K 9/0031; C12N 2310/14; C12N 2320/32; C12N 2320/51

(57) ABSTRACT

The present invention relates to specific reverse micelle system of the invention which allows the administration and intracellular delivery of unmodified oligonucleotide, such as siRNA, targeting one or more genes of the SARS-CoV-2 virus. The reverse micelle system of the invention is thus particularly useful for the treatment of the viral pathology linked to the SARS-CoV-2 virus.

### FIELD OF THE INVENTION

The present invention relates to specific reverse micelle system of the invention which allows the administration and intracellular delivery of unmodified oligonucleotide, such as siRNA, targeting one or more genes of the SARS-CoV-2 virus. The reverse micelle system of the invention is thus particularly useful for the treatment of the viral pathology linked to the SARS-CoV-2 virus.

### BACKGROUND OF THE INVENTION

Coronavirus disease 2019 (COVID-19) is an infectious disease caused by the severe acute respiratory syndrome coronavirus 2 (SARS-CoV-2). The disease has spread globally since 2019, resulting in the 2019-21 coronavirus pandemic. Common symptoms include fever, cough and shortness of breath.

Muscle pain, sputum production and sore throat are less common symptoms. While the majority of cases result in mild symptoms, some progress to pneumonia and multi-organ failure. The deaths per number of diagnosed cases is estimated at between 1% and 5% but varies by age and other health conditions.

There is consequently a major public health emergency to treat CoVID_19 which is spreading worldwide very quickly.

RNAi (RNA interference) and antisense (AS) strategies consist in silencing the expression of a target gene by the use of nucleic acids which allow the degradation or the translational arrest of mRNA target. New antisense applications (exon skipping, alternative splicing correction), by masking the mutation responsible for an alternative splicing default, have permitted the synthesis of a functional protein. Aptamers are nucleic acids capable of interacting with a target protein and down regulating its synthesis. The discovery of all these nucleic acids, and more recently siRNA and miRNA, has opened wide perspectives in therapeutics for the treatment of diseases like genetic diseases, cancers, neurodegenerative diseases, infectious and inflammatory diseases or to block cell proliferation and diseases caused thereby.

However, these molecules are unstable in biological fluids, in vitro and in vivo, they display a poor intracellular penetration and low bioavailability. These critical drawbacks have limited their use in therapeutics. As a result, clinical applications of said nucleic acids have required chemical modifications with the aim of retaining their capacity to knockdown protein expression while increasing stability and cellular penetration. Research groups have also applied the nanotechnology approach to improve their delivery, to overcome most barriers that hampered the development of nucleic acids delivery-based therapies. To improve bioavailability, many researchers have also attempted to use alternative administration routes: ocular, skin, oral, intramuscular. Those attempts have not been totally satisfactory so far. For instance, some of these attempts, more specifically assays with nucleic acids in liposome carriers have stimulated immune response.

The object of the present invention is to overcome disadvantages of the prior art. There is an obvious need for a safe and efficient nucleic acids therapeutic strategy for the treatment of diseases related to SARS-CoV-2 virus (or for the treatment of COVID-19), and in particular for new tools that are able to achieve efficient gene expression modulation-based therapy in order to treat diseases related to SARS-CoV-2 virus. More particularly, it is an object of the invention to provide a drug delivery system comprising an unmodified oligonucleotide targeting one or more genes of SARS-CoV-2, which can be for instance administered via buccal mucosa, giving rise to a satisfactory drug bioavailability in an active form.

### SUMMARY OF THE INVENTION

The present invention relates to a delivery system for the in vivo, or ex vivo release of unmodified oligonucleotides targeting SARS-CoV-2 RNAs, by administration to the buccal or rectal mucosa of said delivery system, as well as the compositions and methods for preparing the delivery system.

More specifically, the delivery system is a reverse micelle system comprising at least one sterol, acylglycerol, phospholipid, an alcohol, and at least one unmodified oligonucleotide targeting one or more genes of SARS-CoV-2 virus.

Herein described are reverse micelle systems designed to reach this goal in a safe and controlled manner.

The reverse micelle systems are able to be absorbed through mucosa and to vectorize unmodified oligonucleotides under a protected form to all cells of any tissue of the organism. The invention also relates to a pharmaceutical composition comprising a reverse micelle system as defined herein and a pharmacologically acceptable support or carrier.

The present invention relates to the use of unmodified oligonucleotides targeting CoV_2019 RNAs, in particular siRNAs whose sequences have been designed to inhibit one or more genes expression of this virus, in the preparation of reverse micelle systems or pharmaceutical compositions comprising the same in the treatment of diseases related to SARS-CoV-2 (or in the treatment of COVID-19).

The aim of the present invention is to provide unmodified oligonucleotides targeting SARS-CoV-2 RNAs in a delivery system that allows to vectorize said oligonucleotides as to down regulate or knock down the expression of a target nucleic acid of SARS-CoV-2 virus, with high efficiency and limited off-target-mediated secondary effects.

Drug delivery technology according to the invention allows intracellular delivery to all tissues and organs using HDL lipoprotein (High Density Lipoprotein) or vHDL lipoprotein (Very-High-Density Lipoprotein) receptors.

More particularly, the present invention provides a reverse-micelle transport system for delivering unmodified oligonucleotides capable of modulation of gene expression or duplication of genes of SARS-CoV-2 virus. More specifically, reverse micelles according to the invention allow the incorporation thereof in HDL and vHDL lipoprotein in the buccal or rectal mucosa. Reverse micelles according to the invention are thus carried in a protected lymphatic transport form, then in the general blood circulation which finally allows an intracellular delivery of said oligonucleotides by the membrane receptors of HDL lipoproteins, of the SRB-1 type (Scavenger receptor class B type 1).

Advantageously, at no time can the subject's immune system detect the presence of the oligonucleotide, with absence of immune reaction when unmodified oligonucleotides are administered in the technology according to the invention.

The reverse micelles can be prepared according to a method described below using at least a sterol, an acylglycerol, a phospholipid, an alcohol, water, and at least one unmodified oligonucleotide targeting one or more genes of SARS-CoV-2 virus.

Said micelles are more particularly obtainable by the following method:
(a) Contacting (i) sterol, preferably sitosterol or cholesterol, (ii) acylglycerol, preferably diacylglycerol of fatty acids, (iii) phospholipid, preferably phosphatidylcholine, (iv) alcohol, (v) water, preferably purified water, and (vi) at least an unmodified oligonucleotide targeting one or more genes of SARS-CoV-2 virus,
(b) Stirring mixture obtained in step (a), at 40° C. or less, and for a time sufficient to obtain formation of reverse micelles, said stirring being preferably carried out mechanically or by sonication.

The parameters of the mechanical stirring, for instance duration and speed, can be readily determined by anyone skilled in the art and depend on experimental conditions. In practice, these parameters are such that a micro-emulsion is obtained; the speed is determined so as to enable formation of a visually transparent formulation, and duration of the stirring is such that the stirring may be stopped a few minutes after obtaining the visually transparent formulation.

The present invention further relates to a pharmaceutical composition comprising reverse micelles of the invention and a pharmaceutically acceptable carrier, excipient or support.

### DETAILED DESCRIPTION OF THE INVENTION

The following description is of preferred embodiments by way of examples only and without limitation to the combination of features necessary for implementing the invention.

Reverse Micelles

The reverse micelle system according to the invention is characterized as a micro-emulsion comprising a dispersion of water-nanodroplets in oil. The dispersion is stabilised by two surfactants (acylglycerol, more preferably a diacylglycerol of fatty acids and a phospholipid, more preferably phosphatidylcholine) and a co-surfactant (alcohol) that are most likely at the water/oil interface. The reverse micelle phase can be defined as a system wherein water forms the internal phase and the hydrophobic tails of the lipids form the continuous phase. Reverse micelles containing oil(s), surfactant(s), co-surfactant(s), and an aqueous phase are also characterized as water-in-oil micro-emulsions.

Generally, the size of micelles according to the invention is very small, more particularly, it is less than 10 nm; more specifically it is less than 8 nm and more preferably less than 5 nm. The size may vary with the quantity of added water and phospholipid. The present invention relates more particularly to reverse micelles with an aqueous core of 3 to 5 nm, preferably from 3.5 to 5 nm, in particular from 3.7 to 4.5 nm.

The reverse micelles and the size of their aqueous core can be characterized by various methods, including:
Small Angle X-Ray Scattering (SAXS)
Neutrons Scattering
Transmission Electron Microscopy (TEM)
Dynamic Light Scattering (DLS)

The ratios of the lipidic constituents (including sterol, acylglycerol and phospholipid) in the reverse-micelle system according to the invention can vary. For instance, the weight ratio sterol/acylglycerol can range from 0.015 to 0.05, more particularly from 0.03 to 0.04. The weight ratio phospholipid/acylglycerol is from 0.06 to 0.25. For the calculation of these ratios, the weight of phospholipid corresponds to the total weight of the mixture of phospholipids, for instance the weight of lecithin, used in the formulation.

The compounds of the reverse-micelle system can be analysed by appropriate means. More specifically, sterols can be identified by gas chromatographic analysis and acylglycerol by high-performance liquid chromatography (HPLC), in particular with a light scattering detector, on a silica column (kromasil C18), in the presence of an eluent, e.g. isocratic acetonitrile. Gas chromatography can also be used to analyse diacylglycerols. Phospholipids can be analysed by high-performance liquid chromatography (HPLC), with a diol column with a light scattering detector.

Reverse micelles are dynamic systems. Brownian motion causes perpetual collisions of micelles, which lead to coalescence of micelles and exchange of the aqueous cores. Separation and regeneration of micelles occur and allow chemical reactions between different solutions. The exchange rate between micelles increases in particular with temperature, the length of hydrocarbon chains of the surfactant, and the water/surfactant ratio. Within the context of the invention and contrary to what is expected in nanotechnology, aqueous cores of micelles must have a specific size allowing one or more molecules of unmodified oligonucleotide, in particular nucleic acid capable of mediating RNA interference, to be stabilised in the prepared micelles. As mentioned above, the size of the aqueous core is around 4 nm, preferably from 3 to 5 nm, more preferably from 3.5 to 5 nm, in particular from 3.7 to 4.5 nm.

Without being bound to any theory, it seems that inclusion of a phospholipid in the reverse micelle system allows formation of micelles with greater diameter and volume, thus allowing vectorization of greater quantities of oligonucleotide.

In addition, it seems that, when applied to mucosa tissue, the reverse micelle system triggers formation of lipoproteins which after a lymphatic transport then in the blood circulation cross the cellular membrane and allow delivery of the oligonucleotide, in particular the nucleic acid capable of modulating gene expression of SARS-CoV-2 virus into the cells.

After the deposition of the micro emulsion on the buccal mucosa (or rectal mucosa) in the subject, the Brownian dynamics of the reverse micelles promotes intramucosal penetration into the intercellular spaces, and in contact with the apoproteins present physiologically in the mucosa, there takes place a structure in lipoproteins vHDL and HDL.

Oligonucleotides must be soluble in water, so as not to interfere with the water/oil interface of the reverse micelles according to the invention.

An amphiphilic molecule modifies the water solubility in the nano micelles, interferes with the interface and removes the fluidity of the permanent Brownian-like motions of the micelles which is necessary for their passage in the mucosa and their absorption through the structuration in lipoproteins.

The oligonucleotides described in the present invention are necessarily unmodified in order to be water-soluble.

Accordingly, the invention ensures absorption of the compounds to be delivered across mucosa, preferably across mouth, nasal and/or rectal mucosa, more preferably across mouth mucosa. Also, reverse micelles of the present invention provide an important bioavailability with low variability of absorption.

Method for Preparing Reverse Micelles

In a particular embodiment, the invention relates to a method for preparing reverse micelles as defined above (involving more specifically at least one unmodified oligonucleotide targeting one or more genes of SARS-CoV-2 virus, a sterol, an acylglycerol, a phospholipid, an alcohol, and water), wherein said method comprises the following steps:
(a) Contacting (i) sterol, (ii) acylglycerol, preferably diacylglycerol of fatty acids, (iii) phospholipid, preferably phosphatidylcholine, (iv) alcohol, (v) water, preferably purified water, and (vi) at least one unmodified oligonucleotide capable of targeting one or more genes of SARS-CoV-2 virus,
(b) Stirring mixture obtained in step (a), at 40° C. or less, and for a time sufficient to obtain formation of reverse micelles, said stirring being carried out mechanically or by sonication.

The obtained and recovered reverse micelles are then particularly useful as a delivery system for unmodified oligonucleotides. Step (b) of the process is of particular importance since it allows reverse micelles to be obtained, said reverse micelles being then useful as a transport system to deliver unmodified oligonucleotides directly into the cytoplasm of all cells in all tissues and organs, through the cell membrane lipoprotein receptors.

In a particular embodiment, the unmodified oligonucleotide is first solubilised in water (preferably purified water) to form an aqueous phase. Said aqueous phase is then introduced into the oily phase (according to step(a)). The oily phase preferably comprises at least a sterol, an acylglycerol, a phospholipid and an alcohol.

The compounds involved in step (a) will be described in more details below.

Stirring of the mixture obtained by step (a) is carried out at a temperature less than or equal to 40° C., preferably ranging from 30° C. to 38° C., more preferably from 30° C. to 35° C., for a time sufficient to form of reverse micelles. The time sufficient can vary in particular upon the used stirring techniques, i.e., mechanical stirring or sonication. The time of mechanical stirring or sonication is more specifically the time needed to convert the initial mixture into a visually transparent reverse micelle solution.

One skilled in the art knows how to select excipients or components that may be used along with the composition according to the present invention in order to keep their beneficial properties. In particular, the presence of glycerol can, when introduced in large amount, prevent the formation of reverse micelles or break the reverse micelle system. More specifically, no more than 2.5%, and preferably no glycerol (percent expressed by weight of glycerol/weight of acylglycerol) is used for the preparation of the reverse micelles of the present invention.

Other compounds can be introduced in step (a). One can cite for instance colouring agents and/or flavouring substances.

In an advantageous manner, the compounds cited above or the commercially available mixtures containing them are the only ingredients introduced to prepare the micelle system and consequently the only ones present in the micelle system of the invention.

Physical parameters, in particular time—for instance comprised between 3 and 5 minutes, in one or several times-, are dependent on the used material, volumes of the mixture and viscosity thereof. One skilled in the art can readily define such parameters. Temperature of the mixture is less than 40° C. Such a temperature avoids degradation of the reactants. Temperature is preferably ranging from 30° C. to 38° C., more preferably from 30° C. to 35° C.

The usual materials use propellers whose fast movements generate turbulences and swirls allowing interpenetration of particles and formation of reverse micelles within the mixture.

Stirring speed is preferably ranging from 200 to 2 000 r/minute, more preferably from 300 to 700 r/minute. The implemented volumes, device, and stirring speed depend on and should be adapted with the reactants and amounts thereof.

As described above, temperature of the mixture must not exceed 40° C. Temperature is preferably ranging from 30° C. to 38° C., more preferably from 30° C. to 35° C.

Reverse Micelles Compounds

Acylglycerol

Acylglycerols, more particularly acylglycerols of fatty acids, useful for the preparation of the reverse-micelle system according to the invention can be isolated from the majority of animals and more preferably plants.

Acylglycerols can be mono- and/or diacylglycerols. In a particular embodiment, mono- or diacylglycerols preferentially used in the present invention present the following formula (I):

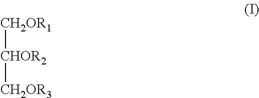

in which:
R₁ is an acyl residue of a linear or branched, saturated or unsaturated fatty acid having between 14 and 24 carbon atoms, a hydrogen atom, or a mono-, di- or tri-galactose or glucose;
R₂ is an acyl residue of a linear or branched, saturated or unsaturated fatty acid having between 2 and 18 carbon atoms;
R₃ is an acyl residue of a linear or branched, saturated or unsaturated fatty acid having between 14 and 24 carbon atoms, or a hydrogen atom.

According to a particular embodiment, R₁ or R₃, preferably only one of R₁ and R₃, in particular only R₁, represents an acyl residue of oleic acid (C18: 1[cis]-9), including in particular glycerol monooleate.

According to a particular aspect, R₂ has one unsaturated bond (e.g; ethylenic bond) and has advantageously 18 carbon atoms, preferably R₂ is an oleic acid residue (oleoyl group), one of its positional isomers with respect to the double bond (cis-6,7,9,11 and 13) or one of its iso-branched isomers.

According to another particular aspect, R₁ represents an oleoyl group.

According to another particular aspect, R₂ represents an acetyl group.

According to another particular aspect, R₃ is a hydrogen atom.

As a general rule, oil containing a high concentration of oleic acid will be chosen as a useful source of acylglycerols according to the invention. Such oil usually contains a high proportion of acylglycerols useful according to the invention.

According to a particular aspect of the invention, the preferred diglycerols of fatty acids are selected in the group consisting of 1,2-diolein and 1-oleoyl-2-acetyl glycerol.

A certain number of them, and more particularly those which are found to be the most active in the applications sought after, are also available commercially. This is the case particularly for 1-oleoyl-2-acetylglycerol and 1,2-dioleoylglycerol, which exist as commercial products with a high purity content. In particular, glycerol monooleate containing about 44% of dioleic glycerol, from which about 14% is 1,2-diolein. Such a compound is pharmaceutically accepted (European Pharmacopeia (4^{th} Edition), USP 25/NF20, and Japanese Standard of food Additives). Such product is for instance commercially available by Gattefossé Company under the name PECEOL®.

The acylglycerols are preferably incorporated or comprised in the composition or reverse-micelle system in an amount by weight ranging from 55 g to 90 g with respect to 100 g of the total weight of the composition or reverse-micelle system according to the invention.

Sterols

The sterols useful for the preparation of the reverse-micelle system according to the invention are preferably natural sterols, such as cholesterol or phytosterols (vegetable sterols). Sitosterol or cholesterol are the preferred sterols useful for the reverse-micelle system according to the invention.

Sitosterol and cholesterol are commercially available. More particularly, commercial sitosterol which is extracted from soya can be used. In such a product, the sitosterol generally represents from 50 to 70% by weight of the product and is generally found in a mixture with campesterol and sitostanol in respective proportions in the order of 15% each. Commercial sitosterol which is extracted from a variety of pine called tall oil can also be used. In general, it will be possible to use sitosterol in mixture with sitostanol. Preferably, said mixture comprises at least 50% sitosterol by weight of the mixture.

As mentioned above, the ratios of the lipidic constituents (sterols, acylglycerol and phospholipids) in the reverse-micelle system according to the invention can vary in a wide range, for instance the weight ratio sterols/acylglycerol can range from 0.015 to 0.05, more particularly from 0.03 to 0.04.

Phospholipids

Phospholipids are formed of a glycerol linked to 2 fatty acids and to a phosphate group. The variability of phospholipids relies on the fatty acids that are attached to the glycerol and on the chemical groups that are susceptible to link to the phosphate group. Phospholipids are the major lipidic constituents of biological membranes.

Among phospholipids useful in the present invention may be cited phosphatidylethanolamine, phosphatidylserine, phosphatidylglycerol, diphosphatidylglycerol, phosphatidylinositol, and phosphatidylcholine.

In a particular embodiment, the phospholipid is phosphatidylcholine. Phosphatidylcholine is also known as 1,2-diacyl-glycero-3-phosphocholine or PtdCho.

Phosphatidylcholine is formed from a choline, a phosphate group, a glycerol and two fatty acids. It is actually a group of molecules, wherein the fatty acid compositions vary from one molecule to another. Phosphatidylcholine may be obtained from commercial lecithin that contains phosphatidylcholine in concentrations of 20 to 98%. The lecithin preferably used for the preparation of the reverse micelles according to the invention is Epikuron 200® and contains phosphatidylcholine at a concentration of more than 90%.

The weight ratio phospholipid/acylglycerol in compositions or reverse-micelle systems according to the invention is from 0.06 to 0.30.

Alcohols

The alcohols useful for the preparation of the reverse-micelle system according to the invention are preferably linear or branched mono-alcohols from C2 to C6. Examples of alcohols are ethanol, 1-butanol, 2-butanol, 3-methyl-1-butanol, 2-methyl-1-propanol, 1-pentanol, 1-propanol, 2-propanol and any mixture thereof. In a particular embodiment of the invention, alcohol is ethanol.

The alcohol is preferably incorporated or comprised in the composition or reverse-micelle system in an amount by weight ranging from 5 g to 17 g with respect to 100 g of the total weight of the composition or reverse-micelle system according to the invention.

Oligonucleotides

The unmodified oligonucleotides targeting SARS-CoV-2 RNAs or targeting one or more genes of SARS-CoV-2 virus can be any nucleic acid molecule capable of modulating gene expression by down regulating or knocking down the expression of a target nucleic acid sequence of SARS-CoV-2 virus.

Down regulating or knocking down the expression of a target nucleic acid sequence can be commonly accomplished via RNA interference (RNAi). RNAi generally designates a phenomenon by which dsRNA specifically reduces expression of a target gene at post-translational level. In normal conditions. RNA interference is initiated by double-stranded RNA molecules (dsRNA) of various length, for example ranging from 15 to 30 base pair length. In vivo, dsRNA introduced into a cell is cleaved into a mixture of short dsRNA molecules.

Nucleic acid molecules capable of modulating gene expression by down regulating or knocking down the expression of a target nucleic acid sequence SARS-CoV-2 virus can thus include “antisense oligonucleotides”, “short interfering nucleic acid” (siNA), “short interfering RNA” (siRNA), “short interfering nucleic acid molecule”, “short interfering oligonucleotide molecule”, “miRNA”, “micro RNA”, guide RNA (gRNA), short guide RNA (sgRNA) of a CRISPR system, “short hairpin RNA” (shRNA) or a mixture thereof.

Unmodified oligonucleotides as defined above, such as unmodified siRNAs, are prone to rapid degradation by ubiquitous endo- and exonucleases and they are generally undetectable in the blood already 10 min after administration.

The oligonucleotides used in the present invention are necessarily chemically unmodified in order to be perfectly water-soluble. More specifically, unmodified oligonucleotides refer to oligonucleotides without any structural modifications at the ribose level (e.g. 2′-fluoro, 2′-methyl, and/or 2′-methoxy), at the base level and at the backbone level (e.g. phosphodiester, phosphorithioate).

According to a preferred embodiment, oligonucleotides of the present invention are at least 10, 15, 20 or 25 nucleotides (nt) long, more preferably in the range of 19 to 25 nucleotides long, or typically 19, 20, 21, 22, 23, 24 or 25 nt long.

According to a preferred embodiment, oligonucleotides of the present invention are designed to have complementarity to the target sequence. In the context of the present invention, they are more specifically designed to have complementarity to a target nucleic acid sequence of the SARS-CoV-2 virus genome. Said viral genome is for instance as described by SEQ ID NO 7.

The term RNA interference (RNAi) is used to describe gene silencing or knocking down at the mRNA level guided by small complementary non-coding RNA species. There are several classes of RNAi mediators, one of which, namely small interfering RNAs (siRNAs). The source of siRNAs during infection is viral double-stranded RNA (dsRNA), which is cleaved by cytoplasmic RNAse III family enzyme Dicer into 19-27 base pair (bp) long molecules with a perfectly complementary middle region and 2-nt overhangs on both 3′ ends. These siRNAs are incorporated into a multiprotein RNA-induced silencing complex (RISC). Following the strand separation, the antisense strand (i.e. guide strand) guides the RISC to recognize and cut target RNA transcripts (the other strand is called passenger strand).

Unmodified oligonucleotides as defined above, such as unmodified siRNAs, are aimed at inhibiting or reducing contagiousness of SARS-CoV-2 virus, more specifically, by protecting host from viral infection, inhibiting the expression of viral antigen or accessory genes, controlling the transcription, retro transcription or replication of viral genome, hindering the assembly of viral particles, or displaying influences in virus-host interactions.

Unmodified oligonucleotides as defined above, such as unmodified siRNAs, can thus be used to protect host from viral infection, inhibit the expression of viral antigen and accessory genes, control the transcription, retro transcription or replication of viral genome, hinder the assembly of viral particles, or display influences in virus-host interactions.

Whether RNAi is a functional antiviral pathway in mammals is still contentious, since production of siRNA molecules from long dsRNAs cannot be explicitly demonstrated in mammalian cells due to the fact that dsRNA longer than 30 bp triggers activation of interferon (IFN) response which shuts down the natural RNAi. However, mammalian cells do possess all the components of evolutionary conserved RNAi machinery that can be harnessed to inhibit the expression of cognate mRNA by exogenous siRNA molecules. The antiviral potential of siRNAs was first demonstrated against respiratory syncytial virus and thereafter numerous studies describing antiviral activity of siRNAs against viruses with DNA and RNA genomes in vitro and in vivo have been published. RNAi-based drugs thus appear to be a viable option to treat severe viral infections, against which effective vaccines or specific cure is not available yet, such as Ebola virus or emerging viruses, in particular SARS-CoV-2 virus.

The first step in production of antiviral siRNAs is in silico selection of highly conservative sequences in the targeted virus genome in order to achieve strong antiviral activity and avoid off-target effects.

After internalization of siRNA duplexes in treated cells, the duplexes are loaded on proteins of the “Ago” family, forming a molecular complex named “RISC” (RNA-induced silencing complex). There are 4 Ago proteins in mouse and in human, but only one (called “Ago2”) is able to degrade target RNAs by endonucleolytic cleavage. That reaction generally occurs when the guide strand and the target are highly complementary (a perfect match to the “seed” [preferably nucleotides 2-7 of the guide strand] is important for target binding; and a perfect match to the central part [preferably nucleotides 8-14] of the guide strand is important for target cleavage).

According to a preferred embodiment, oligonucleotides of the present invention are designed to have complementarity to a target nucleic acid sequence of SARS-CoV-2 virus genome (such as SEQ ID NO 7). This complementarity involves at least 13 bases, typically between 13 and 25 bases, preferably at least 14 bases, even more preferably at least 18 bases of the oligonucleotides of the present invention.

The term “complementary” or “complementarity” refers herein to the ability of oligonucleotides to form base pairs with another nucleotide molecule. Base pairs are typically formed by hydrogen bonds between nucleotide units in antiparallel polynucleotide strands. Complementary polynucleotide strands can base pair in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenosine. However, when a U is denoted in the context of the present invention, the ability to substitute a T is implied, unless otherwise stated. Perfect complementarity or 100 percent complementarity refers to the situation in which each nucleotide unit of the oligonucleotide strand of the invention can bind to a nucleotide unit of a second oligonucleotide strand. Less than perfect complementarity refers to the situation in which some, but not all, nucleotide units of two strands can bind with each other. For example, for two 20-mers, if only two base pairs on one strand of the invention (e.g. guide strand) can bind with the other, the oligonucleotide strand of the invention exhibits 10 percent complementarity. In the same way, if 20 base units of one 20 nt strand (e.g. guide strand) can be bond with 20 other base units of the target gene, the oligonucleotide strands of the invention exhibit 100 percent complementarity.

In a particular aspect, the oligonucleotides of the present invention is a RNA, typically a double-stranded RNA (or RNA duplexes), in particular a small interfering RNA (siRNA), with a guide strand and a passenger strand.

According to a more particular embodiment, the oligonucleotides of the present invention are synthetic RNA duplexes comprising or consisting of two unmodified 21-mer oligonucleotides annealed together to form short/small interfering RNAs (siRNAs).

The main limitation of anti-viral RNAi is the great mutability of viruses. It can be anticipated that siRNA-resistant virus variants will emerge rapidly. In order to delay as much as possible, the emergence of such variants, it is better to target constant regions of the virus genome (i.e.: regions that are exactly identical among the sequenced variants of the virus).

According to a particular embodiment, the unmodified oligonucleotide targeting one or more genes of the virus SARS-CoV-2 targets constant regions of the virus genome.

mRNA accessibility to RISC can be hindered by RNA-binding proteins, whose binding pattern is not known. But the 5′ UTR and coding sequence of mRNAs are cleaned by ribosome scanning making them more sensitive to RISC than the 3′ UTR: while scoring predicted off-targets, it is advisable to focus on those with a seed match in their 3′ UTR.

According to a particular embodiment, the unmodified oligonucleotide targeting one or more genes of the virus SARS-CoV-2 targets the 5′ UTR and coding sequence of the virus genome.

mRNA accessibility to RISC can also be inhibited by mRNA secondary structures, especially short-term interactions, which are likely to re-form rapidly after ribosome scanning.

According to a particular embodiment, the unmodified oligonucleotide targeting one or more genes of the virus SARS-CoV-2 targets poorly-structured regions of the mRNA of SARS-CoV-2 virus.

Natural human miRNAs frequently have a 5′ uridine, which may be due to an intrinsically higher affinity of the Ago protein or its loading machinery (at least Ago2 binds preferentially 5′ uridines and 5′ adenosines).

According to a particular embodiment, the unmodified oligonucleotide targeting one or more genes of SARS-CoV-2 virus, and in particular its guide strand, has a 5′ uridine base.

In addition to the intended target, introduced siRNAs might bind additional mRNAs (“off-targets”). The main determinant of target recognition is a perfect match between nucleotides 2-7 of the guide strand (the “seed” of the guide strand) and the off-target RNA. If there are many off-targets, the siRNA is likely to be partially titrated, hence less efficient. And because off-targets might be (moderately) repressed by the siRNA, they could trigger unwanted secondary effects. It is thus preferable to choose siRNAs that minimize the number of off-targets, and to minimize the number of off-targets whose modest down-regulation is most susceptible to trigger phenotypic consequences in humans.

The SARS-CoV-2 virus has an RNA genome, so it is theoretically possible to target both the genomic RNA (which is about 30 kb long) and individual mRNAs (there are 9 annotated ORFs—open reading frames—in the SARS-CoV-2 genome). mRNAs are more likely to be accessible to siRNAs, because genomic RNA is largely protected by encapsidation.

According to a particular embodiment, the unmodified oligonucleotide targeting one or more genes of SARS-CoV-2 targets one or more viral sequences that belong to mature mRNAs of SARS-CoV-2.

According to a particular embodiment, the unmodified oligonucleotide of the invention targets the mRNA of the longest protein produced by the virus. The function of this protein is not yet known with precision; in the viral genome, its gene is called “ORFlab”. Said gene extends from position 266 to position 21555 of the viral genome shown in SEQ ID NO 7, and siRNA of the invention more preferably targets the region between positions 14790 to 14810 of said genome.

Up to date, these positions of the viral genome are, at the same time, 100% conserved between all the sequenced variants of the virus genome (more than one thousand variants described to date) and they are particularly accessible to siRNAs. (little folded in on themselves).

According to a particular embodiment, the siRNA of the invention presents a guide strand which comprises, or consists of, one of the following sequences:

SEQ ID NO 1:
5′ P-UGAUAGUAGUCAUAAUCGCUA 3′;
SEQ ID NO 3:
5′ P-UGACUUAAAGUUCUUUAUGCG 3′;
SEQ ID NO 5:
5′ P-UUAGCUAAAGACACGAACCGG 3′;
SEQ ID NO 8:
5′ P-UGACUUAAAGUUCUUUAUGCUC 3′;
SEQ ID NO 10:
5′ P-UAUAGCUAAAGACACGAACCC 3′;
SEQ ID NO 11:
5′ P-AUAGCUAAAGACACGAACCGG 3′;
SEQ ID NO 12:
5′ P-UUGAGUGCAUCAUUAUCCAAG 3′;
SEQ ID NO 13:
5′ P-CUUGACUGCCGCCUCUGCUCG 3′;
SEQ ID NO 14:
5′ P-GUUGAGUGCAUCAUUAUCCAC 3′;
SEQ ID NO 15:
5′ P-UCCUGAUUAUGUACAACACCG 3′.

Preferably, the siRNA of the invention presents a guide strand which comprises, or consists of, SEQ ID NO 1.

According to a preferred embodiment, the siRNA duplexes of the invention, with guide strand and passenger strand, comprises, or consists of, one of the following duplex sequences: siRNA n° 1

SEQ ID NO 1: guide strand:
5′ P-UGAUAGUAGUCAUAAUCGCUA 3′;
SEQ ID NO 2: passenger strand:
5′ GCGAUUAUGACUACUAUUUUA 3′.
siRNA no 2
SEQ ID NO 3: guide strand:
5′ P-UGACUUAAAGUUCUUUAUGCG 3′;
SEQ ID NO 4: passenger strand:
5′ CAUAAAGAACUUUAAGUCCUC 3′.
siRNA no 3
SEQ ID NO 5: guide strand:
5′ P-UUAGCUAAAGACACGAACCGG 3′;
SEQ ID NO 6: passenger strand:
5′ GGUUCGUGUCUUUAGCUACUC 3′.
siRNA no 4
SEQ ID NO 8: guide strand:
5′ P-UGACUUAAAGUUCUUUAUGCUC 3′;
SEQ ID NO 9: passenger strand:
5′ GCAUAAAGAACUUUAAGUUUCU 3′.

Preferably, the siRNA of the invention comprises, or consists of, the duplex sequences of SEQ ID NO 1 and 2 (siRNA n° 1).

The siRNA with guide stands corresponding to SEQ ID 10-15 also comprise passenger strands as to form effective siRNAs duplexes, as described above.

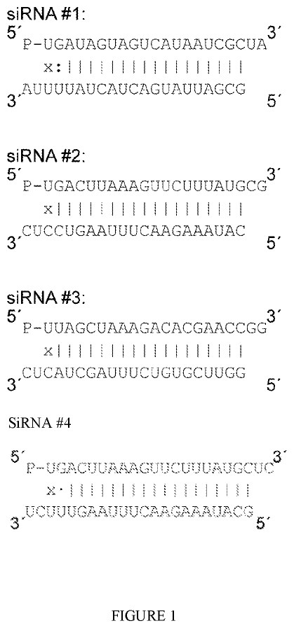

Schematic of said siRNA structures are dispatched in FIG. 1 (|: Watson-Crick base pair, x: mismatch; ′: GU wobble).

According to a particular aspect, the invention relates to the siRNAS as identified above.

According to another aspect, the invention relates a pharmaceutical composition comprising at least one the siRNAs as defined above, in a pharmaceutically acceptable carrier or excipient.

Use of Reverse Micelles to Deliver Unmodified Nucleotides Targeting Virus SARS-Cov-2 Genes

The pharmaceutical composition of the present invention describes unmodified oligonucleotides, such as siRNAs, targeting one or more genes of the SARS-CoV-2 Coronavirus virus, formulated in the microemulsion (or reverse micelles) as described herein and intended for the treatment of patients contaminated by this virus.

The unmodified oligonucleotides, such as siRNAs, targeting one or more genes of the SARS-CoV-2 are present in the aqueous core of the reverse micelles.

The amount of unmodified oligonucleotides, such as siRNAs, targeting one or more genes of SARS-CoV-2 incorporated into the reverse micelle system is determined by their solubility in the hydrophilic phase (aqueous core). Preferably, the amount of unmodified oligonucleotides, such as siRNAs, targeting one or more genes of SARS-CoV-2 included in the reverse micelle system depends on their size.

The reverse micelles of the invention allow the oligonucleotide included therein to be administered and transported to cells with a high degree of protection in lipoprotein HDL and vHDL, in particular without affecting its stability.

It is known today that a reverse-micelle system can be used for the preparation of nanomaterials, which act as micro reactors. The activity and stability of bio molecules can be controlled, mainly by the concentration of water in this medium.

An object of the invention concerns a pharmaceutical composition comprising reverse micelles as defined above and at least a pharmaceutically acceptable carrier, excipient or support, more specifically for use in the treatment of COVID-19.

According to a particular embodiment, the pharmaceutical composition is in the form of airless bottle, a capsule, a caplet, an aerosol, a spray, a solution or a soft elastic gelatin capsule.

A further object of the invention concerns the use of reverse micelles as defined above for preparing a pharmaceutical composition intended for the treatment of COVID-19.

The present invention further concerns a method for the treatment of COVID-19, wherein the method comprises the step of administering into a subject in need of such treatment a therapeutically efficient amount of one or more unmodified oligonucleotides as defined above.

More specifically, administration of one or more unmodified oligonucleotides in reverse micelle system as defined herein or pharmaceutical composition comprising the same is a mucosal delivery.

As pharmaceutically acceptable excipient, vehicle or carrier, any excipient, vehicle or carrier well-known to the person skilled in the art may be used. Other additives well-known to the person skilled in the art such as stabilisers, drying agents, binders or pH buffers may also be used. Preferred excipients in accordance with the invention promote adherence of the finished product to the mucosa.

The compositions of the invention can be administered in different ways, in particular via the oral, nasal, vaginal or rectal route, with a buccal, nasal, vaginal or digestive absorption, or more generally via mucosal tissue absorption. The composition of the invention is preferably administered by buccal route or rectal route, with a buccal mucosa or rectal mucosa absorption, respectively.

Within the context of the invention, the term treatment denotes curative, symptomatic, and preventive treatment. As used herein, the term “treatment” of COVID-19 refers to any act intended to extend life span of subjects (or patients) such as therapy and retardation of the disease progression. The treatment can be designed to eradicate the disease, to stop the progression of the disease, and/or to promote the regression of the disease. The term “treatment” of a disease also refers to any act intended to decrease one or more mild symptoms associated with the disease, including fever, cough, shortness of breath, muscle pain, sputum production and/or sore throat. The term “treatment” of the disease also refers to any act intended to decrease one or more severe symptoms associated with the disease, including pneumonia and/or multi-organ failure. More specifically, the treatment according to the invention is intended to delay the appearance of, alleviate, or hinder, the mild symptoms and more particularly the severe symptoms of COVID-19, such as COVID-19 associated pneumonia or multi-organ failure.

As used herein, the term “therapeutically effective amount” is intended an amount of unmodified oligonucleotides as defined above, administered to a patient that is sufficient to constitute a treatment of COVID-19 as defined above. In a particular embodiment, the therapeutically effective amount to be administered is an amount sufficient to down regulate or knock down the expression of a target nucleic acid of SARS-CoV-2 virus. The amount of unmodified oligonucleotides as defined above to be administered can be determined by standard procedure well known by those of ordinary skill in the art. Physiological data of the patient (e.g. age, size, and weight), the routes of administration and the disease to be treated have to be taken into account to determine the appropriate dosage. One skilled in the art will recognize that the amount of unmodified oligonucleotides to be administered will be an amount that is sufficient to induce reduction of COVID-19 symptoms or to induce alleviation of one or more symptoms of COVID-19.

The subject (or patient) to treat is any mammal, preferably a human being. Preferably, the subject is a human patient, whatever its age or sex. New-borns, infants, children are included as well. More preferably, the patient or subject according to the invention is suspected to be infected by SARS-CoV-2 or has been diagnosed to have CoVID-19 or has been diagnosed as infected by SARS-CoV-2. The standard method of diagnosis is by reverse transcription polymerase chain reaction (rRT-PCR) from a nasopharyngeal swab or throat swab. The infection can also be diagnosed from a combination of symptoms, risk factors and a chest CT scan (Computer Tomography scan) showing features of pneumonia.

As used herein, the terms “mucosa” and “mucosal” refer to a mucous tissue such as of the respiratory, digestive, or genital tissue. “Mucosal delivery”, “mucosal administration” and analogous terms as used herein refer to the administration of a composition through a mucosal tissue. “Mucosal delivery”, “mucosal administration” and analogous terms include, but are not limited to, the delivery of a composition through preferably buccal administration, bronchi, gingival, lingual, nasal, buccal, vaginal, rectal, and gastro-intestinal mucosal tissue. Administration according to the invention is more preferably carried out via buccal mucosa or rectal mucosa.

### EXAMPLES

The following examples are intended to exemplify the operation of the present invention but not to limit its scope.

### Example 1: Manufacture of a Drug for the Treatment of Infectious Pathologies Linked to SARS-CoV-2 Coronavirus—siRNA #1

The aim of this study was to evaluate by visual determination the formulation and the stability of the siRNA #1 targeting SARS-CoV-2 in the reverse microemulsion.

8.5 g of lecithin were dissolved in 6.8 g of absolute ethanol by magnetic stirring at 300 r/min for 15 minutes at room temperature. 1.4 g of sitosterol were added to the mixture and stirred in the same conditions. 32.6 g of glycerol monooleate was added thereto and magnetic stirring was carried out at 500 r/min for 45 minutes at 37° C.

168 mg of a siRNA aqueous solution containing 1.06 mg of siRNA #1 were added to 1148.0 mg of the oil mixture as prepared above and then stirred at room temperature by magnetic stirring at 700 r/min for 30 minutes.

The microemulsion was limpid, monophasic and thermodynamically stable. These experiments show that the siRNA #1 is well formulated in the reverse micelle at 800 sg/ml and has no impact on the stability of the system.

Other microemulsions with close contents are also prepared and result in similar reverse micelle with a stable system.

### Example 2: Manufacture of a Drug for the Treatment of Infectious Pathologies Linked to SARS-CoV-2 Coronavirus—siRNA #2, siRNA #3, siRNA #4

The aim of this study was to evaluate by visual determination the formulation and the stability of the siRNA #2, #3 et #4 targeting SARS-CoV-2 in the reverse microemulsion.

8.5 g of lecithin were dissolved in 6.8 g of absolute ethanol by magnetic stirring at 300 r/min for 15 minutes at room temperature. 1.4 g of sitosterol were added to the mixture and stirred in the same conditions. 32.6 g of glycerol monooleate was added thereto and magnetic stirring was carried out at 500 r/min for 45 minutes at 37° C.

168 mg of a siRNA aqueous solution were added to 1148.0 mg of the oil mixture as prepared above and then stirred at room temperature by magnetic stirring at 700 r/min for 30 minutes. The composition of each tested siRNA aqueous solution is in following table 1:

TABLE 1
tested siRNA aqueous solutions

| #2 | #3 | #4 |
|---|---|---|
| siRNA aqueous | siRNA aqueous | siRNA aqueous |
| solution | solution | solution |
| containing 1.06 mg | containing 1.06 mg of | containing 1.06 mg |
| of siRNA #2 | siRNA #3 | of siRNA #4 |

Each microemulsion obtained was limpid, monophasic and thermodynamically stable. These experiments show that the siRNA #2, siRNA #3 and siRNA #4 are well formulated in the reverse micelles at 800 sg/ml and have no impact on the stability of the systems.

### Example 3: Efficacy Study by Rectal Mucosa Route in SG Hamster Model of SARS-COV-2

Objective:

Evaluation of 2 reverse micelle systems according to the invention (with siRNA #1 and with siRNA #2) in hamster model of SARS-COV-2 after 4-day treatment.

Test Items:

| Name | NanosiRNA ®#1 | NanosiRNA ®#2 | NanosiRNA ®#SCR |
|---|---|---|---|
| Medesis Batch | N° 210006 | N^{o}: 210007 | N^{o}: 210009 |
| Com- | water in oil | water in oil | water in oil |
| position | microemulsion | microemulsion | microemulsion |
| | containing | containing | containing |
| | 1.0 mg/mL | 1.0 mg/mL | 1.0 mg/mL |
| | of siRNA#1, | of siRNA#2, | of scrambled |
| | targeting | targeting | siRNA. |
| | the SARS-Cov-2 | the SARS-Cov-2 | |
| | coronavirus. | coronavirus. | |

| | |
|---|---|
| Description | Homogeneous yellow oily liquid |
| | Viscosity approx. 80-100 mPa · s at +25° C. and |
| | approx 40-60 mPa · s at +37° C.. |
| Storage conditions | at room temperature (approximately +15 to +25° C.). |
| Expiry date | January 2022 |
| Quantity | 6 mL (3 vials of 2 mL) Clear glass vials containing 2 mL of drug product |

Administration:

A constant dosage-volume of 1 mL/kg/day is used for all groups of animals. The quantity of dosage form administered to each animal is adjusted according to the bodyweight.

The dosage form is administrated by rectal deposit without anesthesia, using a pipet tip with automatic pipetman.

Food and water will be removed before product administration and will be given 30 minutes after administration.

Animals:

Strain: Golden Syrian hamster model of SARS-COV-2

Number: 24 female SG hamsters

6-8 weeks old female SG hamsters of 90-120 g are ear-tagged and randomized in the different treatment groups.

| | | P 6 virus | | | | |
|---|---|---|---|---|---|---|
| | | TCID₅₀ | | | Frequency | |
| Group | hamsters | inoculum/50 μL | Treatment | Dose | dosing | MOA |
| Group 1 | 6 WT | 1.89E+06 | siRNA 1 | 1.0 mg/mL | once | Rectal deposit |
| Group 2 | 6 WT | 1.89E+06 | siRNA 2 | 1.0 mg/mL | once | Rectal deposit |
| Group 3 | 6 WT | 1.89E+06 | Scramble | 1.0 mg/mL | once | Rectal deposit |
| | 18 hamsters | | | | | |

Duration:

The dose formulations will be administrated once daily for a period of 4 days.

Collection and Analysing of Samples:

Animals are sacrificed: lung and blood collection

Lung: 1) Quantification of viral load by real-time quantitative RT-qPCR

2) Quantification of infectious viral content by (end-point) titration

3) Histological examination for evaluation of inflammation in lung tissues.

| SEQ ID NO 7: | |
|---|---|
| 1 | attaaaggtt tataccttcc caggtaacaa accaaccaac tttcgatctc ttgtagatct |
| 61 | gttctctaaa cgaactttaa aatctgtgtg gctgtcactc ggctgcatgc ttagtgcact |
| 121 | cacgcagtat aattaataac taattactgt cgttgacagg acacgagtaa ctcgtctatc |
| 181 | ttctgcaggc tgcttacggt ttcgtccgtg ttgcagccga tcatcagcac atctaggttt |
| 241 | cgtccgggtg tgaccgaaag gtaagatgga gagccttgtc cctggtttca acgagaaaac |
| 301 | acacgtccaa ctcagtttgc ctgttttaca ggttcgcgac gtgctcgtac gtggctttgg |
| 361 | agactccgtg gaggaggtct tatcagaggc acgtcaacat cttaaagatg gcacttgtgg |
| 421 | cttagtagaa gttgaaaaag gcgttttgcc tcaacttgaa cagccctatg tgttcatcaa |
| 481 | acgttcggat gctcgaactg cacctcatgg tcatgttatg gttgagctgg tagcagaact |
| 541 | cgaaggcatt cagtacggtc gtagtggtga gacacttggt gtccttgtcc ctcatgtggg |
| 601 | cgaaatacca gtggcttacc gcaaggttct tcttcgtaag aacggtaata aaggagctgg |
| 661 | tggccatagt tacggcgccg atctaaagtc atttgactta ggcgacgagc ttggcactga |
| 721 | tccttatgaa gattttcaag aaaactggaa cactaaacat agcagtggtg ttacccgtga |
| 781 | actcatgcgt gagcttaacg gaggggcata cactcgctat gtcgataaca acttctgtgg |
| 841 | ccctgatggc taccctcttg agtgcattaa agaccttcta gcacgtgctg gtaaagcttc |
| 901 | atgcactttg tccgaacaac tggactttat tgacactaag aggggtgtat actgctgccg |
| 961 | tgaacatgag catgaaattg cttggtacac ggaacgttct gaaaagagct atgaattgca |
| 1021 | gacacctttt gaaattaaat tggcaaagaa atttgacacc ttcaatgggg aatgtccaaa |
| 1081 | ttttgtattt cccttaaatt ccataatcaa gactattcaa ccaagggttg aaaagaaaaa |
| 1141 | gcttgatggc tttatgggta gaattcgatc tgtctatcca gttgcgtcac caaatgaatg |
| 1201 | caaccaaatg tgcctttcaa ctctcatgaa gtgtgatcat tgtggtgaaa cttcatggca |
| 1261 | gacgggcgat tttgttaaag ccacttgcga attttgtggc actgagaatt tgactaaaga |
| 1321 | aggtgccact acttgtggtt acttacccca aaatgctgtt gttaaaattt attgtccagc |
| 1381 | atgtcacaat tcagaagtag gacctgagca tagtcttgcc gaataccata atgaatctgg |
| 1441 | cttgaaaacc attcttcgta agggtggtcg cactattgcc tttggaggct gtgtgttctc |
| 1501 | ttatgttggt tgccataaca agtgtgccta ttgggttcca cgtgctagcg ctaacatagg |
| 1561 | ttgtaaccat acaggtgttg ttggagaagg ttccgaaggt cttaatgaca accttcttga |
| 1621 | aatactccaa aaagagaaag tcaacatcaa tattgttggt gactttaaac ttaatgaaga |
| 1681 | gatcgccatt attttggcat ctttttctgc ttccacaagt gcttttgtgg aaactgtgaa |
| 1741 | aggtttggat tataaagcat tcaaacaaat tgttgaatcc tgtggtaatt ttaaagttac |
| 1801 | aaaaggaaaa gctaaaaaag gtgcctggaa tattggtgaa cagaaatcaa tactgagtcc |
| 1861 | tctttatgca tttgcatcag aggctgctcg tgttgtacga tcaattttct cccgcactct |
| 1921 | tgaaactgct caaaattctg tgcgtgtttt acagaaggcc gctataacaa tactagatgg |
| 1981 | aatttcacag tattcactga gactcattga tgctatgatg ttcacatctg atttggctac |
| 2041 | taacaatcta gttgtaatgg cctacattac aggtggtgtt gttcagttga cttcgcagtg |
| 2101 | gctaactaac atctttggca ctgtttatga aaaactcaaa cccgtccttg attggcttga |
| 2161 | agagaagttt aaggaaggtg tagagtttct tagagacggt tgggaaattg ttaaatttat |
| 2221 | ctcaacctgt gcttgtgaaa ttgtcggtgg acaaattgtc acctgtgcaa aggaaattaa |
| 2281 | ggagagtgtt cagacattct ttaagcttgt aaataaattt ttggctttgt gtgctgactc |
| 2341 | tatcattatt ggtggagcta aacttaaagc cttgaattta ggtgaaacat ttgtcacgca |
| 2401 | ctcaaaggga ttgtacagaa agtgtgttaa atccagagaa gaaactggcc tactcatgcc |
| 2461 | tctaaaagcc ccaaaagaaa ttatcttctt agagggagaa acacttccca cagaagtgtt |
| 2521 | aacagaggaa gttgtcttga aaactggtga tttacaacca ttagaacaac ctactagtga |
| 2581 | agctgttgaa gctccattgg ttggtacacc agtttgtatt aacgggctta tgttgctcga |
| 2641 | aatcaaagac acagaaaagt actgtgccct tgcacctaat atgatggtaa caaacaatac |
| 2701 | cttcacactc aaaggcggtg caccaacaaa ggttactttt ggtgatgaca ctgtgataga |
| 2761 | agtgcaaggt tacaagagtg tgaatatcac ttttgaactt gatgaaagga ttgataaagt |
| 2821 | acttaatgag aagtgctctg cctatacagt tgaactcggt acagaagtaa atgagttcgc |
| 2881 | ctgtgttgtg gcagatgctg tcataaaaac tttgcaacca gtatctgaat tacttacacc |
| 2941 | actgggcatt gatttagatg agtggagtat ggctacatac tacttatttg atgagtctgg |
| 3001 | tgagtttaaa ttggcttcac atatgtattg ttctttctac cctccagatg aggatgaaga |
| 3061 | agaaggtgat tgtgaagaag aagagtttga gccatcaact caatatgagt atggtactga |
| 3121 | agatgattac caaggtaaac ctttggaatt tggtgccact tctgctgctc ttcaacctga |
| 3181 | agaagagcaa gaagaagatt ggttagatga tgatagtcaa caaactgttg gtcaacaaga |
| 3241 | cggcagtgag gacaatcaga caactactat tcaaacaatt gttgaggttc aacctcaatt |
| 3301 | agagatggaa cttacaccag ttgttcagac tattgaagtg aatagtttta gtggttattt |
| 3361 | aaaacttact gacaatgtat acattaaaaa tgcagacatt gtggaagaag ctaaaaaggt |
| 3421 | aaaaccaaca gtggttgtta atgcagccaa tgtttacctt aaacatggag gaggtgttgc |
| 3481 | aggagcctta aataaggcta ctaacaatgc catgcaagtt gaatetgatg attacatagc |
| 3541 | tactaatgga ccacttaaag tgggtggtag ttgtgtttta agcggacaca atcttgctaa |
| 3601 | acactgtctt catgttgtcg gcccaaatgt taacaaaggt gaagacattc aacttcttaa |
| 3661 | gagtgcttat gaaaatttta atcagcacga agttctactt gcaccattat tatcagctgg |
| 3721 | tatttttggt gctgacccta tacattcttt aagagtttgt gtagatactg ttcgcacaaa |
| 3781 | tgtctactta gctgtctttg ataaaaatct ctatgacaaa cttgtttcaa gctttttgga |
| 3841 | aatgaagagt gaaaagcaag ttgaacaaaa gatcgctgag attcctaaag aggaagttaa |
| 3901 | gccatttata actgaaagta aaccttcagt tgaacagaga aaacaagatg ataagaaaat |
| 3961 | caaagcttgt gttgaagaag ttacaacaac tctggaagaa actaagttcc tcacagaaaa |
| 4021 | cttgttactt tatattgaca ttaatggcaa tcttcatcca gattctgcca ctcttgttag |
| 4081 | tgacattgac atcactttct taaagaaaga tgctccatat atagtgggtg atgttgttca |
| 4141 | agagggtgtt ttaactgctg tggttatacc tactaaaaag gctggtggca ctactgaaat |
| 4201 | gctagcgaaa gctttgagaa aagtgccaac agacaattat ataaccactt acccgggtca |
| 4261 | gggtttaaat ggttacactg tagaggaggc aaagacagtg cttaaaaagt gtaaaagtgc |
| 4321 | cttttacatt ctaccatcta ttatctctaa tgagaagcaa gaaattcttg gaactgtttc |
| 4381 | ttggaatttg cgagaaatgc ttgcacatgc agaagaaaca cgcaaattaa tgcctgtctg |
| 4441 | tgtggaaact aaagccatag tttcaactat acagcgtaaa tataagggta ttaaaataca |
| 4501 | agagggtgtg gttgattatg gtgctagatt ttacttttac accagtaaaa caactgtagc |
| 4561 | gtcacttatc aacacactta acgatctaaa tgaaactctt gttacaatgc cacttggcta |
| 4621 | tgtaacacat ggcttaaatt tggaagaagc tgctcggtat atgagatctc tcaaagtgcc |
| 4681 | agctacagtt tctgtttctt cacctgatgc tgttacagcg tataatggtt atcttacttc |
| 4741 | ttcttctaaa acacctgaag aacattttat tgaaaccatc tcacttgctg gttcctataa |
| 4801 | agattggtcc tattctggac aatctacaca actaggtata gaatttctta agagaggtga |
| 4861 | taaaagtgta tattacacta gtaatcctac cacattccac ctagatggtg aagttatcac |
| 4921 | ctttgacaat cttaagacac ttctttcttt gagagaagtg aggactatta aggtgtttac |
| 4981 | aacagtagac aacattaacc tccacacgca agttgtggac atgtcaatga catatggaca |
| 5041 | acagtttggt ccaacttatt tggatggagc tgatgttact aaaataaaac ctcataattc |
| 5101 | acatgaaggt aaaacatttt atgttttacc taatgatgac actctacgtg ttgaggcttt |
| 5161 | tgagtactac cacacaactg atcctagttt tctgggtagg tacatgtcag cattaaatca |
| 5221 | cactaaaaag tggaaatacc cacaagttaa tggtttaact tctattaaat gggcagataa |
| 5281 | caactgttat cttgccactg cattgttaac actccaacaa atagagttga agtttaatcc |
| 5341 | acctgctcta caagatgctt attacagagc aagggctggt gaagctgcta acttttgtgc |
| 5401 | acttatctta gcctactgta ataagacagt aggtgagtta ggtgatgtta gagaaacaat |
| 5461 | gagttacttg tttcaacatg ccaatttaga ttcttgcaaa agagtcttga acgtggtgtg |
| 5521 | taaaacttgt ggacaacagc agacaaccct taagggtgta gaagctgtta tgtacatggg |
| 5581 | cacactttct tatgaacaat ttaagaaagg tgttcagata ccttgtacgt gtggtaaaca |
| 5641 | agctacaaaa tatctagtac aacaggagtc accttttgtt atgatgtcag caccacctgc |
| 5701 | tcagtatgaa cttaagcatg gtacatttac ttgtgctagt gagtacactg gtaattacca |
| 5761 | gtgtggtcac tataaacata taacttctaa agaaactttg tattgcatag acggtgcttt |
| 5821 | acttacaaag tcctcagaat acaaaggtcc tattacggat gttttctaca aagaaaacag |
| 5881 | ttacacaaca accataaaac cagttactta taaattggat ggtgttgttt gtacagaaat |
| 5941 | tgaccctaag ttggacaatt attataagaa agacaattct tatttcacag agcaaccaat |
| 6001 | tgatcttgta ccaaaccaac catatccaaa cgcaagcttc gataatttta agtttgtatg |
| 6061 | tgataatatc aaatttgctg atgatttaaa ccagttaact ggttataaga aacctgcttc |
| 6121 | aagagagctt aaagttacat ttttccctga cttaaatggt gatgtggtgg ctattgatta |
| 6181 | taaacactac acaccctctt ttaagaaagg agctaaattg ttacataaac ctattgtttg |
| 6241 | gcatgttaac aatgcaacta ataaagccac gtataaacca aatacctggt gtatacgttg |
| 6301 | tctttggagc acaaaaccag ttgaaacatc aaattcgttt gatgtactga agtcagagga |
| 6361 | cgcgcaggga atggataatc ttgcctgcga agatctaaaa ccagtctctg aagaagtagt |
| 6421 | ggaaaatcct accatacaga aagacgttct tgagtgtaat gtgaaaacta ccgaagttgt |
| 6481 | aggagacatt atacttaaac cagcaaataa tagtttaaaa attacagaag aggttggcca |
| 6541 | cacagatcta atggctgctt atgtagacaa ttctagtctt actattaaga aacctaatga |
| 6601 | attatctaga gtattaggtt tgaaaaccct tgctactcat ggtttagctg ctgttaatag |
| 6661 | tgtcccttgg gatactatag ctaattatgc taagcctttt cttaacaaag ttgttagtac |
| 6721 | aactactaac atagttacac ggtgtttaaa ccgtgtttgt actaattata tgccttattt |
| 6781 | ctttacttta ttgctacaat tgtgtacttt tactagaagt acaaattcta gaattaaagc |
| 6841 | atctatgccg actactatag caaagaatac tgttaagagt gtcggtaaat tttgtctaga |
| 6901 | ggcttcattt aattatttga agtcacctaa tttttctaaa ctgataaata ttataatttg |
| 6961 | gtttttacta ttaagtgttt gcctaggttc tttaatctac tcaaccgctg ctttaggtgt |
| 7021 | tttaatgtct aatttaggca tgccttctta ctgtactggt tacagagaag gctatttgaa |
| 7081 | ctctactaat gtcactattg caacctactg tactggttct ataccttgta gtgtttgtct |
| 7141 | tagtggttta gattctttag acacctatcc ttctttagaa actatacaaa ttaccatttc |
| 7201 | atcttttaaa tgggatttaa ctgcttttgg cttagttgca gagtggtttt tggcatatat |
| 7261 | tcttttcact aggtttttct atgtacttgg attggctgca atcatgcaat tgtttttcag |
| 7321 | ctattttgca gtacatttta ttagtaattc ttggcttatg tggttaataa ttaatcttgt |
| 7381 | acaaatggcc ccgatttcag ctatggttag aatgtacatc ttctttgcat cattttatta |
| 7441 | tgtatggaaa agttatgtgc atgttgtaga cggttgtaat tcatcaactt gtatgatgtg |
| 7501 | ttacaaacgt aatagagcaa caagagtcga atgtacaact attgttaatg gtgttagaag |
| 7561 | gtccttttat gtctatgcta atggaggtaa aggcttttgc aaactacaca attggaattg |
| 7621 | tgttaattgt gatacattct gtgctggtag tacatttatt agtgatgaag ttgcgagaga |
| 7681 | cttgtcacta cagtttaaaa gaccaataaa tcctactgac cagtcttctt acatcgttga |
| 7741 | tagtgttaca gtgaagaatg gttccatcca tctttacttt gataaagctg gtcaaaagac |
| 7801 | ttatgaaaga cattctctct ctcattttgt taacttagac aacctgagag ctaataacac |
| 7861 | taaaggttca ttgcctatta atgttatagt ttttgatggt aaatcaaaat gtgaagaatc |
| 7921 | atctgcaaaa tcagcgtctg tttactacag tcagcttatg tgtcaaccta tactgttact |
| 7981 | agatcaggca ttagtgtctg atgttggtga tagtgcggaa gttgcagtta aaatgtttga |
| 8041 | tgcttacgtt aatacgtttt catcaacttt taacgtacca atggaaaaac tcaaaacact |
| 8101 | agttgcaact gcagaagctg aacttgcaaa gaatgtgtcc ttagacaatg tcttatctac |
| 8161 | ttttatttca gcagctcggc aagggtttgt tgattcagat gtagaaacta aagatgttgt |
| 8221 | tgaatgtctt aaattgtcac atcaatctga catagaagtt actggcgata gttgtaataa |
| 8281 | ctatatgctc acctataaca aagttgaaaa catgacaccc cgtgaccttg gtgcttgtat |
| 8341 | tgactgtagt gcgcgtcata ttaatgcgca ggtagcaaaa agtcacaaca ttgctttgat |
| 8401 | atggaacgtt aaagatttca tgtcattgtc tgaacaacta cgaaaacaaa tacgtagtgc |
| 8461 | tgctaaaaag aataacttac cttttaagtt gacatgtgca actactagac aagttgttaa |
| 8521 | tgttgtaaca acaaagatag cacttaaggg tggtaaaatt gttaataatt ggttgaagca |
| 8581 | gttaattaaa gttacacttg tgttcctttt tgttgctgct attttctatt taataacacc |
| 8641 | tgttcatgtc atgtctaaac atactgactt ttcaagtgaa atcataggat acaaggctat |
| 8701 | tgatggtggt gtcactcgtg acatagcatc tacagatact tgttttgcta acaaacatgc |
| 8761 | tgattttgac acatggttta gccagcgtgg tggtagttat actaatgaca aagcttgccc |
| 8821 | attgattgct gcagtcataa caagagaagt gggttttgtc gtgcctggtt tgcctggcac |
| 8881 | gatattacgc acaactaatg gtgacttttt gcatttctta cctagagttt ttagtgcagt |
| 8941 | tggtaacatc tgttacacac catcaaaact tatagagtac actgactttg caacatcagc |
| 9001 | ttgtgttttg gctgctgaat gtacaatttt taaagatgct tctggtaagc cagtaccata |
| 9061 | ttgttatgat accaatgtac tagaaggttc tgttgcttat gaaagtttac gccctgacac |
| 9121 | acgttatgtg ctcatggatg gctctattat tcaatttcct aacacctacc ttgaaggttc |
| 9181 | tgttagagtg gtaacaactt ttgattctga gtactgtagg cacggcactt gtgaaagatc |
| 9241 | agaagctggt gtttgtgtat ctactagtgg tagatgggta cttaacaatg attattacag |
| 9301 | atctttacca ggagttttct gtggtgtaga tgctgtaaat ttacttacta atatgtttac |
| 9361 | accactaatt caacctattg gtgctttgga catatcagca tctatagtag ctggtggtat |
| 9421 | tgtagctatc gtagtaacat gccttgccta ctattttatg aggtttagaa gagcttttgg |
| 9481 | tgaatacagt catgtagttg cctttaatac tttactattc cttatgtcat tcactgtact |
| 9541 | ctgtttaaca ccagtttact cattcttacc tggtgtttat tctgttattt acttgtactt |
| 9601 | gacattttat cttactaatg atgtttcttt tttagcacat attcagtgga tggttatgtt |
| 9661 | cacaccttta gtacctttct ggataacaat tgcttatatc atttgtattt ccacaaagca |
| 9721 | tttctattgg ttctttagta attacctaaa gagacgtgta gtctttaatg gtgtttcctt |
| 9781 | tagtactttt gaagaagctg cgctgtgcac ctttttgtta aataaagaaa tgtatctaaa |
| 9841 | gttgcgtagt gatgtgctat tacctcttac gcaatataat agatacttag ctctttataa |
| 9901 | taagtacaag tattttagtg gagcaatgga tacaactagc tacagagaag ctgcttgttg |
| 9961 | tcatctcgca aaggctctca atgacttcag taactcaggt tctgatgttc tttaccaacc |
| 10021 | accacaaacc tctatcacct cagctgtttt gcagagtggt tttagaaaaa tggcattccc |
| 10081 | atctggtaaa gttgagggtt gtatggtaca agtaacttgt ggtacaacta cacttaacgg |
| 10141 | tctttggctt gatgacgtag tttactgtcc aagacatgtg atctgcacct ctgaagacat |
| 10201 | gcttaaccct aattatgaag atttactcat tcgtaagtct aatcataatt tcttggtaca |
| 10261 | ggctggtaat gttcaactca gggttattgg acattctatg caaaattgtg tacttaagct |
| 10321 | taaggttgat acagccaatc ctaagacacc taagtataag tttgttcgca ttcaaccagg |
| 10381 | acagactttt tcagtgttag cttgttacaa tggttcacca tctggtgttt accaatgtgc |
| 10441 | tatgaggccc aatttcacta ttaagggttc attccttaat ggttcatgtg gtagtgttgg |
| 10501 | ttttaacata gattatgact gtgtctcttt ttgttacatg caccatatgg aattaccaac |
| 10561 | tggagttcat gctggcacag acttagaagg taacttttat ggaccttttg ttgacaggca |
| 10621 | aacagcacaa gcagctggta cggacacaac tattacagtt aatgttttag cttggttgta |
| 10681 | cgctgctgtt ataaatggag acaggtggtt tctcaatcga tttaccacaa ctcttaatga |
| 10741 | ctttaacctt gtggctatga agtacaatta tgaacctcta acacaagacc atgttgacat |
| 10801 | actaggacct ctttctgctc aaactggaat tgccgtttta gatatgtgtg cttcattaaa |
| 10861 | agaattactg caaaatggta tgaatggacg taccatattg ggtagtgctt tattagaaga |
| 10921 | tgaatttaca ccttttgatg ttgttagaca atgctcaggt gttactttcc aaagtgcagt |
| 10981 | gaaaagaaca atcaagggta cacaccactg gttgttactc acaattttga cttcactttt |
| 11041 | agttttagtc cagagtactc aatggtcttt gttctttttt ttgtatgaaa atgccttttt |
| 11101 | accttttgct atgggtatta ttgctatgtc tgcttttgca atgatgtttg tcaaacataa |
| 11161 | gcatgcattt ctctgtttgt ttttgttacc ttctcttgcc actgtagctt attttaatat |
| 11221 | ggtctatatg cctgctagtt gggtgatgcg tattatgaca tggttggata tggttgatac |
| 11281 | tagtttgtct ggttttaagc taaaagactg tgttatgtat gcatcagctg tagtgttact |
| 11341 | aatccttatg acagcaagaa ctgtgtatga tgatggtgct aggagagtgt ggacacttat |
| 11401 | gaatgtcttg acactcgttt ataaagttta ttatggtaat gctttagatc aagccatttc |
| 11461 | catgtgggct cttataatct ctgttacttc taactactca ggtgtagtta caactgtcat |
| 11521 | gtttttggcc agaggtattg tttttatgtg tgttgagtat tgccctattt tcttcataac |
| 11581 | tggtaataca cttcagtgta taatgctagt ttattgtttc ttaggctatt tttgtacttg |
| 11641 | ttactttggc ctcttttgtt tactcaaccg ctactttaga ctgactcttg gtgtttatga |
| 11701 | ttacttagtt tctacacagg agtttagata tatgaattca cagggactac tcccacccaa |
| 11761 | gaatagcata gatgccttca aactcaacat taaattgttg ggtgttggtg gcaaaccttg |
| 11821 | tatcaaagta gccactgtac agtctaaaat gtcagatgta aagtgcacat cagtagtctt |
| 11881 | actctcagtt ttgcaacaac tcagagtaga atcatcatct aaattgtggg ctcaatgtgt |
| 11941 | ccagttacac aatgacattc tcttagctaa agatactact gaagcctttg aaaaaatggt |
| 12001 | ttcactactt tctgttttgc tttccatgca gggtgctgta gacataaaca agctttgtga |
| 12061 | agaaatgctg gacaacaggg caaccttaca agctatagcc tcagagttta gttcccttcc |
| 12121 | atcatatgca gcttttgcta ctgctcaaga agcttatgag caggctgttg ctaatggtga |
| 12181 | ttctgaagtt gttcttaaaa agttgaagaa gtctttgaat gtggctaaat ctgaatttga |
| 12241 | ccgtgatgca gccatgcaac gtaagttgga aaagatggct gatcaagcta tgacccaaat |
| 12301 | gtataaacag gctagatctg aggacaagag ggcaaaagtt actagtgcta tgcagacaat |
| 12361 | gcttttcact atgcttagaa agttggataa tgatgcactc aacaacatta tcaacaatgc |
| 12421 | aagagatggt tgtgttccct tgaacataat acctcttaca acagcagcca aactaatggt |
| 12481 | tgtcatacca gactataaca catataaaaa tacgtgtgat ggtacaacat ttacttatgc |
| 12541 | atcagcattg tgggaaatcc aacaggttgt agatgcagat agtaaaattg ttcaacttag |
| 12601 | tgaaattagt atggacaatt cacctaattt agcatggcct cttattgtaa cagctttaag |
| 12661 | ggccaattct gctgtcaaat tacagaataa tgagcttagt cctgttgcac tacgacagat |
| 12721 | gtcttgtgct gccggtacta cacaaactgc ttgcactgat gacaatgcgt tagcttacta |
| 12781 | caacacaaca aagggaggta ggtttgtact tgcactgtta tccgatttac aggatttgaa |
| 12841 | atgggctaga ttccctaaga gtgatggaac tggtactatc tatacagaac tggaaccacc |
| 12901 | ttgtaggttt gttacagaca cacctaaagg tcctaaagtg aagtatttat actttattaa |
| 12961 | aggattaaac aacctaaata gaggtatggt acttggtagt ttagctgcca cagtacgtct |
| 13021 | acaagctggt aatgcaacag aagtgcctgc caattcaact gtattatctt tctgtgcttt |
| 13081 | tgctgtagat gctgctaaag cttacaaaga ttatctagct agtgggggac aaccaatcac |
| 13141 | taattgtgtt aagatgttgt gtacacacac tggtactggt caggcaataa cagttacacc |
| 13201 | ggaagccaat atggatcaag aatcctttgg tggtgcatcg tgttgtctgt actgccgttg |
| 13261 | ccacatagat catccaaatc ctaaaggatt ttgtgactta aaaggtaagt atgtacaaat |
| 13321 | acctacaact tgtgctaatg accctgtggg ttttacactt aaaaacacag tctgtaccgt |
| 13381 | ctgcggtatg tggaaaggtt atggctgtag ttgtgatcaa ctccgcgaac ccatgcttca |
| 13441 | gtcagctgat gcacaatcgt ttttaaacgg gtttgcggtg taagtgcagc ccgtcttaca |
| 13501 | ccgtgcggca caggcactag tactgatgtc gtatacaggg cttttgacat ctacaatgat |
| 13561 | aaagtagctg gttttgctaa attcctaaaa actaattgtt gtcgcttcca agaaaaggac |
| 13621 | gaagatgaca atttaattga ttcttacttt gtagttaaga gacacacttt ctctaactac |
| 13681 | caacatgaag aaacaattta taatttactt aaggattgtc cagctgttgc taaacatgac |
| 13741 | ttctttaagt ttagaataga cggtgacatg gtaccacata tatcacgtca acgtcttact |
| 13801 | aaatacacaa tggcagacct cgtctatgct ttaaggcatt ttgatgaagg taattgtgac |
| 13861 | acattaaaag aaatacttgt cacatacaat tgttgtgatg atgattattt caataaaaag |
| 13921 | gactggtatg attttgtaga aaacccagat atattacgcg tatacgccaa ettaggtgaa |
| 13981 | cgtgtacgcc aagctttgtt aaaaacagta caattctgtg atgccatgcg aaatgctggt |
| 14041 | attgttggtg tactgacatt agataatcaa gatctcaatg gtaactggta tgatttcggt |
| 14101 | gatttcatac aaaccacgcc aggtagtgga gttcctgttg tagattctta ttattcattg |
| 14161 | ttaatgccta tattaacctt gaccagggct ttaactgcag agtcacatgt tgacactgac |
| 14221 | ttaacaaagc cttacattaa gtgggatttg ttaaaatatg acttcacgga agagaggtta |
| 14281 | aaactctttg accgttattt taaatattgg gatcagacat accacccaaa ttgtgttaac |
| 14341 | tgtttggatg acagatgcat tctgcattgt gcaaacttta atgttttatt ctctacagtg |
| 14401 | ttcccaccta caagttttgg accactagtg agaaaaatat ttgttgatgg tgttccattt |
| 14461 | gtagtttcaa ctggatacca cttcagagag ctaggtgttg tacataatca ggatgtaaac |
| 14521 | ttacatagct ctagacttag ttttaaggaa ttacttgtgt atgctgctga ccctgctatg |
| 14581 | cacgctgctt ctggtaatct attactagat aaacgcacta cgtgcttttc agtagctgca |
| 14641 | cttactaaca atgttgcttt tcaaactgtc aaacccggta attttaacaa agacttctat |
| 14701 | gactttgctg tgtctaaggg tttctttaag gaaggaagtt ctgttgaatt aaaacacttc |
| 14761 | ttctttgctc aggatggtaa tgctgctatc agcgattatg actactatcg ttataatcta |
| 14821 | ccaacaatgt gtgatatcag acaactacta tttgtagttg aagttgttga taagtacttt |
| 14881 | gattgttacg atggtggctg tattaatgct aaccaagtca tcgtcaacaa cctagacaaa |
| 14941 | tcagctggtt ttccatttaa taaatggggt aaggctagac tttattatga ttcaatgagt |
| 15001 | tatgaggatc aagatgcact tttcgcatat acaaaacgta atgtcatccc tactataact |
| 15061 | caaatgaatc ttaagtatgc cattagtgca aagaatagag ctcgcaccgt agctggtgtc |
| 15121 | tctatctgta gtactatgac caatagacag tttcatcaaa aattattgaa atcaatagcc |
| 15181 | gccactagag gagctactgt agtaattgga acaagcaaat tctatggtgg ttggcacaac |
| 15241 | atgttaaaaa ctgtttatag tgatgtagaa aaccctcacc ttatgggttg ggattatcct |
| 15301 | aaatgtgata gagccatgcc taacatgctt agaattatgg cctcacttgt tcttgctcgc |
| 15361 | aaacatacaa cgtgttgtag cttgtcacac cgtttctata gattagctaa tgagtgtgct |
| 15421 | caagtattga gtgaaatggt catgtgtggc ggttcactat atgttaaacc aggtggaacc |
| 15481 | tcatcaggag atgccacaac tgcttatgct aatagtgttt ttaacatttg tcaagctgtc |
| 15541 | acggccaatg ttaatgcact tttatctact gatggtaaca aaattgccga taagtatgtc |
| 15601 | cgcaatttac aacacagact ttatgagtgt ctctatagaa atagagatgt tgacacagac |
| 15661 | tttgtgaatg agttttacgc atatttgcgt aaacatttct caatgatgat actctctgac |
| 15721 | gatgctgttg tgtgtttcaa tagcacttat gcatctcaag gtctagtggc tagcataaag |
| 15781 | aactttaagt cagttcttta ttatcaaaac aatgttttta tgtctgaagc aaaatgttgg |
| 15841 | actgagactg accttactaa aggacctcat gaattttgct ctcaacatac aatgctagtt |
| 15901 | aaacagggtg atgattatgt gtaccttcct tacccagatc catcaagaat cctaggggcc |
| 15961 | ggctgttttg tagatgatat cgtaaaaaca gatggtacac ttatgattga acggttcgtg |
| 16021 | tctttagcta tagatgctta cccacttact aaacatccta atcaggagta tgctgatgtc |
| 16081 | tttcatttgt acttacaata cataagaaag ctacatgatg agttaacagg acacatgtta |
| 16141 | gacatgtatt ctgttatgct tactaatgat aacacttcaa ggtattggga acctgagttt |
| 16201 | tatgaggcta tgtacacacc gcatacagtc ttacaggctg ttggggcttg tgttctttgc |
| 16261 | aattcacaga cttcattaag atgtggtgct tgcatacgta gaccattctt atgttgtaaa |
| 16321 | tgctgttacg accatgtcat atcaacatca cataaattag tcttgtctgt taatccgtat |
| 16381 | gtttgcaatg ctccaggttg tgatgtcaca gatgtgactc aactttactt aggaggtatg |
| 16441 | agctattatt gtaaatcaca taaaccaccc attagttttc cattgtgtgc taatggacaa |
| 16501 | gtttttggtt tatataaaaa tacatgtgtt ggtagcgata atgttactga ctttaatgca |
| 16561 | attgcaacat gtgactggac aaatgctggt gattacattt tagctaacac ctgtactgaa |
| 16621 | agactcaagc tttttgcagc agaaacgctc aaagctactg aggagacatt taaactgtct |
| 16681 | tatggtattg ctactgtacg tgaagtgctg tctgacagag aattacatct ttcatgggaa |
| 16741 | gttggtaaac ctagaccacc acttaaccga aattatgtct ttactggtta tcgtgtaact |
| 16801 | aaaaacagta aagtacaaat aggagagtac acctttgaaa aaggtgacta tggtgatgct |
| 16861 | gttgtttacc gaggtacaac aacttacaaa ttaaatgttg gtgattattt tgtgctgaca |
| 16921 | tcacatacag taatgccatt aagtgcacct acactagtgc cacaagagca ctatgttaga |
| 16981 | attactggct tatacccaac actcaatatc tcagatgagt tttctagcaa tgttgcaaat |
| 17041 | tatcaaaagg ttggtatgca aaagtattct acactccagg gaccacctgg tactggtaag |
| 17101 | agtcattttg ctattggcct agctctctac tacccttctg ctcgcatagt gtatacagct |
| 17161 | tgctctcatg ccgctgttga tgcactatgt gagaaggcat taaaatattt gcctatagat |
| 17221 | aaatgtagta gaattatacc tgcacgtgct cgtgtagagt gttttgataa attcaaagtg |
| 17281 | aattcaacat tagaacagta tgtcttttgt actgtaaatg cattgcctga gacgacagca |
| 17341 | gatatagttg tctttgatga aatttcaatg gccacaaatt atgatttgag tgttgtcaat |
| 17401 | gccagattac gtgctaagca ctatgtgtac attggcgacc ctgctcaatt acctgcacca |
| 17461 | cgcacattgc taactaaggg cacactagaa ccagaatatt tcaattcagt gtgtagactt |
| 17521 | atgaaaacta taggtccaga catgttcctc ggaacttgtc ggcgttgtcc tgctgaaatt |
| 17581 | gttgacactg tgagtgcttt ggtttatgat aataagctta aagcacataa agacaaatca |
| 17641 | gctcaatgct ttaaaatgtt ttataagggt gttatcacgc atgatgtttc atctgcaatt |
| 17701 | aacaggccac aaataggcgt ggtaagagaa ttccttacac gtaaccctgc ttggagaaaa |
| 17761 | gctgtcttta tttcacctta taattcacag aatgctgtag cctcaaagat tttgggacta |
| 17821 | ccaactcaaa ctgttgattc atcacagggc tcagaatatg actatgtcat attcactcaa |
| 17881 | accactgaaa cagctcactc ttgtaatgta aacagattta atgttgctat taccagagca |
| 17941 | aaagtaggca tactttgcat aatgtctgat agagaccttt atgacaagtt gcaatttaca |
| 18001 | agtcttgaaa ttccacgtag gaatgtggca actttacaag ctgaaaatgt aacaggactc |
| 18061 | tttaaagatt gtagtaaggt aatcactggg ttacatccta cacaggcacc tacacacctc |
| 18121 | agtgttgaca ctaaattcaa aactgaaggt ttatgtgttg acatacctgg catacctaag |
| 18181 | gacatgacct atagaagact catctctatg atgggtttta aaatgaatta tcaagttaat |
| 18241 | ggttacccta acatgtttat cacccgcgaa gaagctataa gacatgtacg tgcatggatt |
| 18301 | ggcttcgatg tcgaggggtg tcatgctact agagaagctg ttggtaccaa tttaccttta |
| 18361 | cagctaggtt tttctacagg tgttaaccta gttgctgtac ctacaggtta tgttgataca |
| 18421 | cctaataata cagatttttc cagagttagt gctaaaccac cgcctggaga tcaatttaaa |
| 18481 | cacctcatac cacttatgta caaaggactt ccttggaatg tagtgcgtat aaagattgta |
| 18541 | caaatgttaa gtgacacact taaaaatctc tctgacagag tcgtatttgt cttatgggca |
| 18601 | catggctttg agttgacatc tatgaagtat tttgtgaaaa taggacctga gcgcacctgt |
| 18661 | tgtctatgtg atagacgtgc cacatgcttt tccactgctt cagacactta tgcctgttgg |
| 18721 | catcattcta ttggatttga ttacgtctat aatccgttta tgattgatgt tcaacaatgg |
| 18781 | ggttttacag gtaacctaca aagcaaccat gatctgtatt gtcaagtcca tggtaatgca |
| 18841 | catgtagcta gttgtgatgc aatcatgact aggtgtctag ctgtccacga gtgctttgtt |
| 18901 | aagcgtgttg actggactat tgaatatcct ataattggtg atgaactgaa gattaatgcg |
| 18961 | gcttgtagaa aggttcaaca catggttgtt aaagctgcat tattagcaga caaattccca |
| 19021 | gttcttcacg acattggtaa ccctaaagct attaagtgtg tacctcaagc tgatgtagaa |
| 19081 | tggaagttct atgatgcaca gccttgtagt gacaaagctt ataaaataga agaattattc |
| 19141 | tattcttatg ccacacattc tgacaaattc acagatggtg tatgcctatt ttggaattgc |
| 19201 | aatgtcgata gatatcctgc taattccatt gtttgtagat ttgacactag agtgctatct |
| 19261 | aaccttaact tgcctggttg tgatggtggc agtttgtatg taaataaaca tgcattccac |
| 19321 | acaccagctt ttgataaaag tgcttttgtt aatttaaaac aattaccatt tttctattac |
| 19381 | tctgacagtc catgtgagtc tcatggaaaa caagtagtgt cagatataga ttatgtacca |
| 19441 | ctaaagtctg ctacgtgtat aacacgttgc aatttaggtg gtgctgtctg tagacatcat |
| 19501 | gctaatgagt acagattgta tctcgatgct tataacatga tgatctcagc tggctttagc |
| 19561 | ttgtgggttt acaaacaatt tgatacttat aacctctgga acacttttac aagacttcag |
| 19621 | agtttagaaa atgtggcttt taatgttgta aataagggac actttgatgg acaacagggt |
| 19681 | gaagtaccag tttctatcat taataacact gtttacacaa aagttgatgg tgttgatgta |
| 19741 | gaattgtttg aaaataaaac aacattacct gttaatgtag catttgagct ttgggctaag |
| 19801 | cgcaacatta aaccagtacc agaggtgaaa atactcaata atttgggtgt ggacattgct |
| 19861 | gctaatactg tgatctggga ctacaaaaga gatgctccag cacatatatc tactattggt |
| 19921 | gtttgttcta tgactgacat agccaagaaa ccaactgaaa cgatttgtgc accactcact |
| 19981 | gtcttttttg atggtagagt tgatggtcaa gtagacttat ttagaaatgc ccgtaatggt |
| 20041 | gttcttatta cagaaggtag tgttaaaggt ttacaaccat ctgtaggtcc caaacaagct |
| 20101 | agtcttaatg gagtcacatt aattggagaa gccgtaaaaa cacagttcaa ttattataag |
| 20161 | aaagttgatg gtgttgtcca acaattacct gaaacttact ttactcagag tagaaattta |
| 20221 | caagaattta aacccaggag tcaaatggaa attgatttct tagaattagc tatggatgaa |
| 20281 | ttcattgaac ggtataaatt agaaggctat gccttcgaac atatcgttta tggagatttt |
| 20341 | agtcatagtc agttaggtgg tttacatcta ctgattggac tagctaaacg ttttaaggaa |
| 20401 | tcaccttttg aattagaaga ttttattcct atggacagta cagttaaaaa ctatttcata |
| 20461 | acagatgcgc aaacaggttc atctaagtgt gtgtgttctg ttattgattt attacttgat |
| 20521 | gattttgttg aaataataaa atcccaagat ttatctgtag tttctaaggt tgtcaaagtg |
| 20581 | actattgact atacagaaat ttcatttatg ctttggtgta aagatggcca tgtagaaaca |
| 20641 | ttttacccaa aattacaatc tagtcaagcg tggcaaccgg gtgttgctat gcctaatctt |
| 20701 | tacaaaatgc aaagaatgct attagaaaag tgtgaccttc aaaattatgg tgatagtgca |
| 20761 | acattaccta aaggcataat gatgaatgtc gcaaaatata ctcaactgtg tcaatattta |
| 20821 | aacacattaa cattagctgt accctataat atgagagtta tacattttgg tgctggttct |
| 20881 | gataaaggag ttgcaccagg tacagctgtt ttaagacagt ggttgcctac gggtacgctg |
| 20941 | cttgtcgatt cagatcttaa tgactttgtc tctgatgcag attcaacttt gattggtgat |
| 21001 | tgtgcaactg tacatacagc taataaatgg gatctcatta ttagtgatat gtacgaccct |
| 21061 | aagactaaaa atgttacaaa agaaaatgac tctaaagagg gttttttcac ttacatttgt |
| 21121 | gggtttatac aacaaaagct agctcttgga ggttccgtgg ctataaagat aacagaacat |
| 21181 | tcttggaatg ctgatcttta taagctcatg ggacacttcg catggtggac agcctttgtt |
| 21241 | actaatgtga atgcgtcatc atctgaagca tttttaattg gatgtaatta tcttggcaaa |
| 21301 | ccacgcgaac aaatagatgg ttatgtcatg catgcaaatt acatattttg gaggaataca |
| 21361 | aatccaattc agttgtcttc ctattcttta tttgacatga gtaaatttcc ccttaaatta |
| 21421 | aggggtactg ctgttatgtc tttaaaagaa ggtcaaatca atgatatgat tttatctctt |
| 21481 | cttagtaaag gtagacttat aattagagaa aacaacagag ttgttatttc tagtgatgtt |
| 21541 | cttgttaaca actaaacgaa caatgtttgt ttttcttgtt ttattgccac tagtctctag |
| 21601 | tcagtgtgtt aatcttacaa ccagaactca attaccccct gcatacacta attctttcac |
| 21661 | acgtggtgtt tattaccctg acaaagtttt cagatcctca gttttacatt caactcagga |
| 21721 | cttgttctta cctttctttt ccaatgttac ttggttccat gctatacatg tctctgggac |
| 21781 | caatggtact aagaggtttg ataaccctgt cctaccattt aatgatggtg tttattttgc |
| 21841 | ttccactgag aagtctaaca taataagagg ctggattttt ggtactactt tagattcgaa |
| 21901 | gacccagtcc ctacttattg ttaataacgc tactaatgtt gttattaaag tctgtgaatt |
| 21961 | tcaattttgt aatgatccat ttttgggtgt ttattaccac aaaaacaaca aaagttggat |
| 22021 | ggaaagtgag ttcagagttt attctagtgc gaataattgc acttttgaat atgtctctca |
| 22081 | gccttttctt atggaccttg aaggaaaaca gggtaatttc aaaaatctta gggaatttgt |
| 22141 | gtttaagaat attgatggtt attttaaaat atattctaag cacacgccta ttaatttagt |
| 22201 | gcgtgatctc cctcagggtt tttcggcttt agaaccattg gtagatttgc caataggtat |
| 22261 | taacatcact aggtttcaaa ctttacttgc tttacataga agttatttga ctcctggtga |
| 22321 | ttcttcttca ggttggacag ctggtgctgc agcttattat gtgggttatc ttcaacctag |
| 22381 | gacttttcta ttaaaatata atgaaaatgg aaccattaca gatgctgtag actgtgcact |
| 22441 | tgaccctctc tcagaaacaa agtgtacgtt gaaatccttc actgtagaaa aaggaatcta |
| 22501 | tcaaacttct aactttagag tccaaccaac agaatctatt gttagatttc ctaatattac |
| 22561 | aaacttgtgc ccttttggtg aagtttttaa cgccaccaga tttgcatctg tttatgcttg |
| 22621 | gaacaggaag agaatcagca actgtgttgc tgattattct gtcctatata attccgcatc |
| 22681 | attttccact tttaagtgtt atggagtgtc tcctactaaa ttaaatgatc tctgctttac |
| 22741 | taatgtctat gcagattcat ttgtaattag aggtgatgaa gtcagacaaa tcgctccagg |
| 22801 | gcaaactgga aagattgctg attataatta taaattacca gatgatttta caggctgcgt |
| 22861 | tatagcttgg aattctaaca atcttgattc taaggttggt ggtaattata attacctgta |
| 22921 | tagattgttt aggaagtcta atctcaaacc ttttgagaga gatatttcaa ctgaaatcta |
| 22981 | tcaggccggt agcacacctt gtaatggtgt tgaaggtttt aattgttact ttcctttaca |
| 23041 | atcatatggt ttccaaccca ctaatggtgt tggttaccaa ccatacagag tagtagtact |
| 23101 | ttcttttgaa cttctacatg caccagcaac tgtttgtgga cctaaaaagt ctactaattt |
| 23161 | ggttaaaaac aaatgtgtca atttcaactt caatggttta acaggcacag gtgttcttac |
| 23221 | tgagtctaac aaaaagtttc tgcctttcca acaatttggc agagacattg ctgacactac |
| 23281 | tgatgctgtc cgtgatccac agacacttga gattcttgac attacaccat gttcttttgg |
| 23341 | tggtgtcagt gttataacac caggaacaaa tacttctaac caggttgctg ttctttatca |
| 23401 | ggatgttaac tgcacagaag tccctgttgc tattcatgca gatcaactta ctcctacttg |
| 23461 | gcgtgtttat tctacaggtt ctaatgtttt tcaaacacgt gcaggctgtt taataggggc |
| 23521 | tgaacatgtc aacaactcat atgagtgtga catacccatt ggtgcaggta tatgcgctag |
| 23581 | ttatcagact cagactaatt ctcctcggcg ggcacgtagt gtagctagtc aatccatcat |
| 23641 | tgcctacact atgtcacttg gtgcagaaaa ttcagttgct tactctaata actctattgc |
| 23701 | catacccaca aattttacta ttagtgttac cacagaaatt ctaccagtgt ctatgaccaa |
| 23761 | gacatcagta gattgtacaa tgtacatttg tggtgattca actgaatgca gcaatctttt |
| 23821 | gttgcaatat ggcagttttt gtacacaatt aaaccgtgct ttaactggaa tagctgttga |
| 23881 | acaagacaaa aacacccaag aagtttttgc acaagtcaaa caaatttaca aaacaccacc |
| 23941 | aattaaagat tttggtggtt ttaatttttc acaaatatta ccagatccat caaaaccaag |
| 24001 | caagaggtca tttattgaag atctactttt caacaaagtg acacttgcag atgctggctt |
| 24061 | catcaaacaa tatggtgatt gccttggtga tattgctgct agagacctca tttgtgcaca |
| 24121 | aaagtttaac ggccttactg ttttgccacc tttgctcaca gatgaaatga ttgctcaata |
| 24181 | cacttctgca ctgttagcgg gtacaatcac ttctggttgg acctttggtg caggtgctgc |
| 24241 | attacaaata ccatttgcta tgcaaatggc ttataggttt aatggtattg gagttacaca |
| 24301 | gaatgttctc tatgagaacc aaaaattgat tgccaaccaa tttaatagtg ctattggcaa |
| 24361 | aattcaagac tcactttctt ccacagcaag tgcacttgga aaacttcaag atgtggtcaa |
| 24421 | ccaaaatgca caagctttaa acacgcttgt taaacaactt agctccaatt ttggtgcaat |
| 24481 | ttcaagtgtt ttaaatgata tcctttcacg tcttgacaaa gttgaggctg aagtgcaaat |
| 24541 | tgataggttg atcacaggca gacttcaaag tttgcagaca tatgtgactc aacaattaat |
| 24601 | tagagctgca gaaatcagag cttctgctaa tcttgctgct actaaaatgt cagagtgtgt |
| 24661 | acttggacaa tcaaaaagag ttgatttttg tggaaagggc tatcatctta tgtccttccc |
| 24721 | tcagtcagca cctcatggtg tagtcttctt gcatgtgact tatgtccctg cacaagaaaa |
| 24781 | gaacttcaca actgctcctg ccatttgtca tgatggaaaa gcacactttc ctcgtgaagg |
| 24841 | tgtctttgtt tcaaatggca cacactggtt tgtaacacaa aggaattttt atgaaccaca |
| 24901 | aatcattact acagacaaca catttgtgtc tggtaactgt gatgttgtaa taggaattgt |
| 24961 | caacaacaca gtttatgatc ctttgcaacc tgaattagac tcattcaagg aggagttaga |
| 25021 | taaatatttt aagaatcata catcaccaga tgttgattta ggtgacatct ctggcattaa |
| 25081 | tgcttcagtt gtaaacattc aaaaagaaat tgaccgcctc aatgaggttg ccaagaattt |
| 25141 | aaatgaatct ctcatcgatc tccaagaact tggaaagtat gagcagtata taaaatggcc |
| 25201 | atggtacatt tggctaggtt ttatagctgg cttgattgcc atagtaatgg tgacaattat |
| 25261 | gctttgctgt atgaccagtt gctgtagttg tctcaagggc tgttgttctt gtggatcctg |
| 25321 | ctgcaaattt gatgaagacg actctgagcc agtgctcaaa ggagtcaaat tacattacac |
| 25381 | ataaacgaac ttatggattt gtttatgaga atcttcacaa ttggaactgt aactttgaag |
| 25441 | caaggtgaaa tcaaggatgc tactccttca gattttgttc gcgctactgc aacgataccg |
| 25501 | atacaagcct cactcccttt cggatggctt attgttggcg ttgcacttct tgctgttttt |
| 25561 | cagagcgctt ccaaaatcat aaccctcaaa aagagatggc aactagcact ctccaagggt |
| 25621 | gttcactttg tttgcaactt gctgttgttg tttgtaacag tttactcaca ccttttgctc |
| 25681 | gttgctgctg gccttgaagc cccttttctc tatctttatg ctttagtcta cttcttgcag |
| 25741 | agtataaact ttgtaagaat aataatgagg ctttggcttt gctggaaatg ccgttccaaa |
| 25801 | aacccattac tttatgatgc caactatttt ctttgctggc atactaattg ttacgactat |
| 25861 | tgtatacctt acaatagtgt aacttcttca attgtcatta cttcaggtga tggcacaaca |
| 25921 | agtcctattt ctgaacatga ctaccagatt ggtggttata ctgaaaaatg ggaatctgga |
| 25981 | gtaaaagact gtgttgtatt acacagttac ttcacttcag actattacca gctgtactca |
| 26041 | actcaattga gtacagacac tggtgttgaa catgttacct tcttcatcta caataaaatt |
| 26101 | gttgatgagc ctgaagaaca tgtccaaatt cacacaatcg acggttcatc cggagttgtt |
| 26161 | aatccagtaa tggaaccaat ttatgatgaa ccgacgacga ctactagcgt gcctttgtaa |
| 26221 | gcacaagctg atgagtacga acttatgtac tcattcgttt cggaagagac aggtacgtta |
| 26281 | atagttaata gcgtacttct ttttcttgct ttcgtggtat tcttgctagt tacactagcc |
| 26341 | atccttactg cgcttcgatt gtgtgcgtac tgctgcaata ttgttaacgt gagtcttgta |
| 26401 | aaaccttctt tttacgttta ctctcgtgtt aaaaatctga attcttctag agttcctgat |
| 26461 | cttctggtct aaacgaacta aatattatat tagtttttct gtttggaact ttaattttag |
| 26521 | ccatggcaga ttccaacggt actattaccg ttgaagagct taaaaagctc cttgaacaat |
| 26581 | ggaacctagt aataggtttc ctattcctta catggatttg tcttctacaa tttgcctatg |
| 26641 | ccaacaggaa taggtttttg tatataatta agttaatttt cctctggctg ttatggccag |
| 26701 | taactttagc ttgttttgtg cttgctgctg tttacagaat aaattggatc accggtggaa |
| 26761 | ttgctatcgc aatggcttgt cttgtaggct tgatgtggct cagctacttc attgcttctt |
| 26821 | tcagactgtt tgcgcgtacg cgttccatgt ggtcattcaa tccagaaact aacattcttc |
| 26881 | tcaacgtgcc actccatggc actattctga ccagaccgct tctagaaagt gaactcgtaa |
| 26941 | tcggagctgt gatccttcgt ggacatcttc gtattgctgg acaccatcta ggacgctgtg |
| 27001 | acatcaagga cctgcctaaa gaaatcactg ttgctacatc acgaacgctt tcttattaca |
| 27061 | aattgggagc ttcgcagcgt gtagcaggtg actcaggttt tgctgcatac agtcgctaca |
| 27121 | ggattggcaa ctataaatta aacacagacc attccagtag cagtgacaat attgctttgc |
| 27181 | ttgtacagta agtgacaaca gatgtttcat ctcgttgact ttcaggttac tatagcagag |
| 27241 | atattactaa ttattatgag gacttttaaa gtttccattt ggaatcttga ttacatcata |
| 27301 | aacctcataa ttaaaaattt atctaagtca ctaactgaga ataaatattc tcaattagat |
| 27361 | gaagagcaac caatggagat tgattaaacg aacatgaaaa ttattctttt cttggcactg |
| 27421 | ataacactcg ctacttgtga gctttatcac taccaagagt gtgttagagg tacaacagta |
| 27481 | cttttaaaag aaccttgctc ttctggaaca tacgagggca attcaccatt tcatcctcta |
| 27541 | gctgataaca aatttgcact gacttgcttt agcactcaat ttgcttttgc ttgtcctgac |
| 27601 | ggcgtaaaac acgtctatca gttacgtgcc agatcagttt cacctaaact gttcatcaga |
| 27661 | caagaggaag ttcaagaact ttactctcca atttttctta ttgttgcggc aatagtgttt |
| 27721 | ataacacttt gcttcacact caaaagaaag acagaatgat tgaactttca ttaattgact |
| 27781 | tctatttgtg ctttttagcc tttctgctat tccttgtttt aattatgctt attatctttt |
| 27841 | ggttctcact tgaactgcaa gatcataatg aaacttgtca cgcctaaacg aacatgaaat |
| 27901 | ttcttgtttt cttaggaatc atcacaactg tagctgcatt tcaccaagaa tgtagtttac |
| 27961 | agtcatgtac tcaacatcaa ccatatgtag ttgatgaccc gtgtcctatt cacttctatt |
| 28021 | ctaaatggta tattagagta ggagctagaa aatcagcacc tttaattgaa ttgtgcgtgg |
| 28081 | atgaggctgg ttctaaatca cccattcagt acatcgatat cggtaattat acagtttcct |
| 28141 | gtttaccttt tacaattaat tgccaggaac ctaaattggg tagtcttgta gtgcgttgtt |
| 28201 | cgttctatga agacttttta gagtatcatg acgttcgtgt tgttttagat ttcatctaaa |
| 28261 | cgaacaaact aaaatgtctg ataatggacc ccaaaatcag cgaaatgcac cccgcattac |
| 28321 | gtttggtgga ccctcagatt caactggcag taaccagaat ggagaacgca gtggggcgcg |
| 28381 | atcaaaacaa cgtcggcccc aaggtttacc caataatact gcgtcttggt tcaccgctct |
| 28441 | cactcaacat ggcaaggaag accttaaatt ccctcgagga caaggcgttc caattaacac |
| 28501 | caatagcagt ccagatgacc aaattggcta ctaccgaaga gctaccagac gaattcgtgg |
| 28561 | tggtgacggt aaaatgaaag atctcagtcc aagatggtat ttctactacc taggaactgg |
| 28621 | gccagaagct ggacttccct atggtgctaa caaagacggc atcatatggg ttgcaactga |
| 28681 | gggagccttg aatacaccaa aagatcacat tggcacccgc aatcctgcta acaatgctgc |
| 28741 | aatcgtgcta caacttcctc aaggaacaac attgccaaaa ggcttctacg cagaagggag |
| 28801 | cagaggcggc agtcaagcct cttctcgttc ctcatcacgt agtcgcaaca gttcaagaaa |
| 28861 | ttcaactcca ggcagcagta ggggaacttc tcctgctaga atggctggca atggcggtga |
| 28921 | tgctgctctt gctttgctgc tgcttgacag attgaaccag cttgagagca aaatgtctgg |
| 28981 | taaaggccaa caacaacaag gccaaactgt cactaagaaa tctgctgctg aggcttctaa |
| 29041 | gaagcctcgg caaaaacgta ctgccactaa agcatacaat gtaacacaag ctttcggcag |
| 29101 | acgtggtcca gaacaaaccc aaggaaattt tggggaccag gaactaatca gacaaggaac |
| 29161 | tgattacaaa cattggccgc aaattgcaca atttgccccc agcgcttcag cgttcttcgg |
| 29221 | aatgtcgcgc attggcatgg aagtcacacc ttcgggaacg tggttgacct acacaggtgc |
| 29281 | catcaaattg gatgacaaag atccaaattt caaagatcaa gtcattttgc tgaataagca |
| 29341 | tattgacgca tacaaaacat tcccaccaac agagcctaaa aaggacaaaa agaagaaggc |
| 29401 | tgatgaaact caagccttac cgcagagaca gaagaaacag caaactgtga ctcttcttcc |
| 29461 | tgctgcagat ttggatgatt tctccaaaca attgcaacaa tccatgagca gtgctgactc |
| 29521 | aactcaggcc taaactcatg cagaccacac aaggcagatg ggctatataa acgttttcgc |
| 29581 | ttttccgttt acgatatata gtctactctt gtgcagaatg aattctcgta actacatagc |
| 29641 | acaagtagat gtagttaact ttaatctcac atagcaatct ttaatcagtg tgtaacatta |
| 29701 | gggaggactt gaaagagcca ccacattttc accgaggcca cgcggagtac gatcgagtgt |
| 29761 | acagtgaaca atgctaggga gagctgccta tatggaagag ccctaatgtg taaaattaat |
| 29821 | tttagtagtg ctatccccat gtgattttaa tagcttctta ggagaatgac aaaaaaaaaa |
| 29881 | aaaaaaaaaa aaaaaaaaaa aaa |

1. A reverse micelle system comprising at least one sterol, acylglycerol, phospholipid, an alcohol, water and at least one unmodified oligonucleotide targeting one or more genes of SARS-CoV-2 virus.

2. The reverse micelle system according to claim 1, wherein the micelles present aqueous cores of around 4 nm.

3. The reverse micelle system according to claim 1, wherein acylglycerol presents the following formula (I): 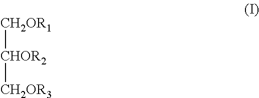
wherein
R₁ is an acyl residue of a linear or branched, saturated or unsaturated fatty acid having between 14 and 24 carbon atoms, a hydrogen atom, or a mono-, di- or tri-galactose or glucose;
R₂ is an acyl residue of a linear or branched, saturated or unsaturated fatty acid having between 2 and 18 carbon atoms; and
R₃ is an acyl residue of a linear or branched, saturated or unsaturated fatty acid having between 14 and 24 carbon atoms, or a hydrogen atom.

4. The reverse micelle system according to claim 1, wherein the at least one sterol is sitosterol, and/or phospholipid is lecithin, and/or alcohol is ethanol, and/or acylglycerol is glycerol monooleate.

5. The reverse micelle system according to claim 1, wherein the unmodified oligonucleotides are selected in the group consisting of antisense oligonucleotides, short interfering nucleic acid (siNA), short interfering RNA (siRNA), short interfering nucleic acid molecule, short interfering oligonucleotide molecule, miRNA, micro RNA, guide RNA (gRNA), short guide RNA (sgRNA) of a CRISPR system, short hairpin RNA (shRNA) and mixtures thereof.

6. The reverse micelle system according to claim 1, wherein the unmodified oligonucleotides are at least 10, 15, 20 or 25 nucleotides (nt) long.

7. The reverse micelle system according to claim 1, wherein the unmodified oligonucleotides are synthetic RNA duplexes comprising or consisting of two unmodified 21-mer oligonucleotides annealed together to form siRNAs.

8. The reverse micelle system according to claim 8, wherein the siRNA presents a guide strand which comprises, or consists of, one of the following sequences:
SEQ ID NO: 1:
5′ P-UGAUAGUAGUCAUAAUCGCUA 3′;
SEQ ID NO: 3:
5′ P-UGACUUAAAGUUCUUUAUGCG 3′;
SEQ ID NO: 5:
5′ P-UUAGCUAAAGACACGAACCGG 3′.
SEQ ID NO: 8:
5′ P-UGACUUAAAGUUCUUUAUGCUC 3′;
SEQ ID NO: 10:
5′ P-UAUAGCUAAAGACACGAACCC 3′;
SEQ ID NO: 11:
5′ P-AUAGCUAAAGACACGAACCGG 3′;
SEQ ID NO: 12:
5′ P-UUGAGUGCAUCAUUAUCCAAG 3′;
SEQ ID NO: 13:
5′ P-CUUGACUGCCGCCUCUGCUCG 3′;
SEQ ID NO: 14:
5′ P-GUUGAGUGCAUCAUUAUCCAC 3′;
or
SEQ ID NO: 15:
5′ P-UCCUGAUUAUGUACAACACCG 3′.[[;]]

9. The reverse micelle system according to claim 8, wherein the siRNA presents a guide strand comprising, or consisting of, SEO ID NO: 1.

10. A method for the preparation of the reverse micelle system according to claim 1, comprising the following steps of:
(a) contacting (i) sterol, (ii) acylglycerol, preferably diacylglycerol of fatty acids, (iii) phospholipid, preferably phosphatidylcholine, (iv) alcohol, (v) water, preferably purified water, and (vi) at least one unmodified oligonucleotide capable of targeting one or more genes of SARS-CoV-2 virus to form a mixture, and
(b) stirring the mixture obtained in step (a), at 40° C. or less, and for a time sufficient to obtain formation of reverse micelles, said stirring being carried out mechanically or by sonication.

11. A pharmaceutical composition comprising a reverse micelle system according to claim 1, and at least a pharmaceutically acceptable carrier, excipient or support.

12. A method for the treatment of COVID-19 comprising administering a therapeutically effective amount of the composition according to claim 11 to a subject in need thereof.

13. A method for the treatment of COVID-19 associated pneumonia or multi-organ failure comprising administering a therapeutically effective amount of the composition according to claim 11 to a subject in need thereof.

14. The method according to claim 12, wherein the composition is administered by oral route or rectal route, with a buccal mucosa or rectal mucosa absorption, respectively.

15. A siRNA presenting a guide strand which comprises, or consists of, one of the following sequences:
SEQ ID NO: 1: guide strand:
5′ P-UGAUAGUAGUCAUAAUCGCUA 3′;
SEQ ID NO: 3: guide strand:
5′ P-UGACUUAAAGUUCUUUAUGCG 3′;
SEQ ID NO: 5: guide strand:
5′ P-UUAGCUAAAGACACGAACCGG 3′;[[.]]
SEQ ID NO: 8: guide strand:
5′ P-UGACUUAAAGUUCUUUAUGCUC 3′;
SEQ ID NO: 10: guide strand:
5′ P-UAUAGCUAAAGACACGAACCC 3′;
SEQ ID NO: 11: guide strand:
5′ P-AUAGCUAAAGACACGAACCGG 3′;
SEQ ID NO: 12: guide strand:
5′ P-UUGAGUGCAUCAUUAUCCAAG 3′;
SEQ ID NO: 13: guide strand:
5′ P-CUUGACUGCCGCCUCUGCUCG 3′;
SEQ ID NO: 14: guide strand:
5′ P-GUUGAGUGCAUCAUUAUCCAC 3′;
or
SEQ ID NO: 15: guide strand:
5′ P-UCCUGAUUAUGUACAACACCG 3′.[[;]]

16. A siRNA duplex, with guide strand and passenger strand, which comprises, or consists of, one of the following duplex sequences:
siRNA no 1
SEQ ID NO: 1: guide strand:
5′ P-UGAUAGUAGUCAUAAUCGCUA 3′;
SEQ ID NO: 2: passenger strand:
5′ GCGAUUAUGACUACUAUUUUA 3′;[[.]]
siRNA no 2
SEQ ID NO: 3: guide strand:
5′ P-UGACUUAAAGUUCUUUAUGCC 3′;
SEQ ID NO: 4: passenger strand:
5′ CAUAAAGAACUUUAAGUCCUC 3′;[[.]]
siRNA no 3
SEQ ID NO: 5: guide strand:
5′ P-UUAGCUAAAGACACGAACCGG 3′;
SEQ ID NO: 6: passenger strand:
5′ GGUUCGUGUCUUUAGCUACUC 3′;[[.]]
or
siRNA no 4
SEQ ID NO: 8: guide strand:
5′ P-UGACUUAAAGUUCUUUAUGCUC 3′;
SEQ ID NO: 9: passenger strand:
5′ GCAUAAAGAACUUUAAGUUUCU 3′.

17. A pharmaceutical composition comprising at least one the siRNAs according to claim 15, and a pharmaceutically acceptable carrier or excipient.

18. The reverse micelle system according to claim 1, wherein the micelles present aqueous cores is from 3 to 5 nm.

19. The reverse micelle system according to claim 6, wherein the unmodified oligonucleotides are in the range of 19 to 25 nucleotides long.

20. A pharmaceutical composition comprising at least one the siRNAs according to claim 16, and a pharmaceutically acceptable carrier or excipient.
